SEQUENCE LISTING (273 sequences)

SEQ ID NO: 1 - DNA, 52 bp, Artificial Sequence
  Synthetic
  misc_structure (1)..(2): phosphorothioate linkage
  misc_structure (2)..(2): phosphorothioate linkage
  misc_structure (50)..(51): phosphorothioate linkage
  misc_structure (51)..(52): phosphorothioate linkage
tcgttcgttccgctctaaccggcgaatctaccgcgcatatctacgccgcaat

SEQ ID NO: 2 - DNA, 52 bp, Artificial Sequence
  Synthetic
  misc_structure (1)..(1): phosphorothioate linkage
  misc_structure (2)..(3): phosphorothioate linkage
  misc_structure (50)..(51): phosphorothioate linkage
  misc_structure (51)..(52): phosphorothioate linkage
attgcggcgtagatatgcgcggtagattcgccggttagagcggaacgaacga

SEQ ID NO: 3 - DNA, 57 bp, Artificial Sequence
  Synthetic
  misc_structure (52)..(52): Ribonucleotide G52
  misc_structure (57)..(57): 3′-C3 spacer
acactctttccctacacgacgctcttccgatcttctaccgcgcatatctacgccgct

SEQ ID NO: 4 - DNA, 58 bp, Artificial Sequence
  Synthetic
  misc_structure (53)..(53): Ribonucleotide C53
  misc_structure (58)..(58): 3′-C3 spacer
acactctttccctacacgacgctcttccgatctatatgcgcggtagattcgccggttt

SEQ ID NO: 5 - DNA, 68 bp, Artificial Sequence
  Synthetic
  misc_structure (63)..(63): Ribonucleotide C63
  misc_structure (68)..(68): 3′-C3 Spacer
gtgactggagttcagacgtgtgctcttccgatctaatgatacggcgaccaccgagatctacacaaggc

SEQ ID NO: 6 - DNA, 78 bp, Artificial Sequence
  Synthetic
  misc_feature (38)..(39): n is a, c, g, or t
  misc_feature (40)..(41): w is a or t
  misc_feature (42)..(42): n is a, c, g, or t
  misc_feature (43)..(44): w is a or t
  misc_feature (45)..(45): n is a, c, g, or t
  misc_structure (76)..(77): phosphorothioate linkage
aatgatacggcgaccaccgagatctacactagatcgcnnwnnwnnacactctttccctacacgacgctcttccgatct

SEQ ID NO: 7 - DNA, 33 bp, Artificial Sequence
  Synthetic
acactctttccctacacgacgctcttccgatct

SEQ ID NO: 8 - DNA, 34 bp, Artificial Sequence
  Synthetic
gtgactggagttcagacgtgtgctcttccgatct

SEQ ID NO: 9 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
acgagcggtagtcacctagtcgtcgtaccaattcgacgcacactactcgcgc

SEQ ID NO: 10 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
gcgcgagtagtgtgcgtcgaattggtacgacgactaggtgactaccgctcgt

SEQ ID NO: 11 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
tagcgcgagtagtcggacgagcggttaccaatacgccgcaccttaatccgcg

SEQ ID NO: 12 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
cgcggattaaggtgcggcgtattggtaaccgctcgtccgactactcgcgcta

SEQ ID NO: 13 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
tcgcgacagtagtcgttcggctaggtacctattaccgcgtagttagcggcgt

SEQ ID NO: 14 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
acgccgctaactacgcggtaataggtacctagccgaacgactactgtcgcga

SEQ ID NO: 15 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
cgcgctactaggtgcgtcgaattggtaccgatccgcaatacactactcgcgc

SEQ ID NO: 16 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
gcgcgagtagtgtattgcggatcggtaccaattcgacgcacctagtagcgcg

SEQ ID NO: 17 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(52): phosphorothioate linkage
  misc_structure (51)..(52): phosphorothioate linkage
ggtaacgagcggtgcgtcgaattggtaaccgctcgtccgaccttaatcgcgc

SEQ ID NO: 18 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
gcgcgattaaggtcggacgagcggttaccaattcgacgcaccgctcgttacc

SEQ ID NO: 19 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
ttcggcgctaggtgcggcgtattggtaaccgctcgtccgttcggcgctaggt

SEQ ID NO: 20 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
acctagcgccgaacggacgagcggttaccaatacgccgcacctagcgccgaa

SEQ ID NO: 21 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
tacgcgactaggtgcgcgattaaggtacctattaccgcgcgactatgtgcgc

SEQ ID NO: 22 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
gcgcacatagtcgcgcggtaataggtaccttaatcgcgcacctagtcgcgta

SEQ ID NO: 23 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
gtcgcgcagtgtagcgcgattaaggtacctattaccgcgtcgcgacagtagt

SEQ ID NO: 24 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
actactgtcgcgacgcggtaataggtaccttaatcgcgctacactgcgcgac

SEQ ID NO: 25 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
aaccgtcgatccgcgcgtagtatggtaccgatccgcaatactagcgcgacaa

SEQ ID NO: 26 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
ttgtcgcgctagtattgcggatcggtaccatactacgcgcggatcgacggtt

SEQ ID NO: 27 - DNA, 52 bp, Artificial Sequence
  Synthetic
  misc_structure (1)..(1): 5′-phosphate
  misc_structure (1)..(2): phosphorothioate linkage
  misc_structure (2)..(2)
  misc_structure (50)..(51): phosphorothioate linkage
  misc_structure (51)..(52): phosphorothioate linkage
tcgctcgattggttacgcgcactacttatgcgctcgactcgttcggctaggt

SEQ ID NO: 28 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
acctagccgaacgagtcgagcgcataagtagtgcgcgtaaccaatcgagcga

SEQ ID NO: 29 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
actgcgagcgtacttgtcgcgctagtaccaattcgacgcaaccgctcgtccg

SEQ ID NO: 30 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
cggacgagcggttgcgtcgaattggtactagcgcgacaagtacgctcgcagt

SEQ ID NO: 31 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
cgcattagtcggtgcggcgtattggtaaccgctcgtccgacgcgctacctat

SEQ ID NO: 32 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
ataggtagcgcgtcggacgagcggttaccaatacgccgcaccgactaatgcg

SEQ ID NO: 33 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
attgcggatcggtgcgtcgaattggtaaccgctcgtccgtacgcgcactact

SEQ ID NO: 34 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
agtagtgcgcgtacggacgagcggttaccaattcgacgcaccgatccgcaat

SEQ ID NO: 35 - DNA, 52 bp, Artificial Sequence
  Synthetic
  misc_structure (1)..(1): 5′-phosphate
  misc_structure (1)..(3): phosphorothioate linkage
  misc_structure (2)..(3): phosphorothioate linkage
  misc_structure (50)..(51): phosphorothioate linkage
  misc_structure (51)..(52): phosphorothioate linkage
tcggcgagtagttgcgcggttatggtaccataaccgcgcagtagtacgcggt

SEQ ID NO: 36 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
accgcgtactactgcgcggttatggtaccataaccgcgcaactactcgccga

SEQ ID NO: 37 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
actagcgatcggtacctagcgccgaaacctattaccgcgacctagcgttgcg

SEQ ID NO: 38 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
cgcaacgctaggtcgcggtaataggtttcggcgctaggtaccgatcgctagt

SEQ ID NO: 39 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
tagcgcgtcaagagcgcggttatggtttcggcgctaggttaacagcgcgtcg

SEQ ID NO: 40 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
cgacgcgctgttaacctagcgccgaaaccataaccgcgctcttgacgcgcta

SEQ ID NO: 41 - DNA, 34 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (32)..(33): phosphorothioate linkage
  misc_structure (33)..(34): phosphorothioate linkage
gtttaattgagttgtcatatgttaataacggtat

SEQ ID NO: 42 - DNA, 34 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (32)..(33): phosphorothioate linkage
  misc_structure (33)..(34): phosphorothioate linkage
ataccgttattaacatatgacaactcaattaaac

SEQ ID NO: 43 - DNA, 20 bp, Artificial Sequence
  Synthetic
gagtccgagcagaagaagaa

SEQ ID NO: 44 - DNA, 20 bp, Artificial Sequence
  Synthetic
gttggagcatctgagtccag

SEQ ID NO: 45 - DNA, 52 bp, Artificial Sequence; identical to SEQ ID NO: 9
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage

SEQ ID NO: 46 - DNA, 52 bp, Artificial Sequence; identical to SEQ ID NO: 11
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage

SEQ ID NO: 47 - DNA, 52 bp, Artificial Sequence; identical to SEQ ID NO: 13
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage

SEQ ID NO: 48 - DNA, 52 bp, Artificial Sequence; identical to SEQ ID NO: 15
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage

SEQ ID NO: 49 - DNA, 52 bp, Artificial Sequence; identical to SEQ ID NO: 17
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage

SEQ ID NO: 50 - DNA, 52 bp, Artificial Sequence; identical to SEQ ID NO: 19
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage

SEQ ID NO: 51 - DNA, 52 bp, Artificial Sequence; identical to SEQ ID NO: 21
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage

SEQ ID NO: 52 - DNA, 52 bp, Artificial Sequence; identical to SEQ ID NO: 23
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage

SEQ ID NO: 53 - DNA, 52 bp, Artificial Sequence; identical to SEQ ID NO: 25
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage

SEQ ID NO: 54 - DNA, 52 bp, Artificial Sequence; identical to SEQ ID NO: 27
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage

SEQ ID NO: 55 - DNA, 52 bp, Artificial Sequence; identical to SEQ ID NO: 29
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage

SEQ ID NO: 56 - DNA, 52 bp, Artificial Sequence; identical to SEQ ID NO: 31
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage

SEQ ID NO: 57 - DNA, 52 bp, Artificial Sequence; identical to SEQ ID NO: 33
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage

SEQ ID NO: 58 - DNA, 52 bp, Artificial Sequence; identical to SEQ ID NO: 35
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage

SEQ ID NO: 59 - DNA, 52 bp, Artificial Sequence; identical to SEQ ID NO: 37
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage

SEQ ID NO: 60 - DNA, 52 bp, Artificial Sequence; identical to SEQ ID NO: 39
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage

SEQ ID NO: 61 - DNA, 52 bp, Artificial Sequence; identical to SEQ ID NO: 10
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage

SEQ ID NO: 62 - DNA, 52 bp, Artificial Sequence; identical to SEQ ID NO: 12
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage

SEQ ID NO: 63 - DNA, 52 bp, Artificial Sequence; identical to SEQ ID NO: 14
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage

SEQ ID NO: 64 - DNA, 52 bp, Artificial Sequence; identical to SEQ ID NO: 16
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage

SEQ ID NO: 65 - DNA, 52 bp, Artificial Sequence; identical to SEQ ID NO: 18
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage

SEQ ID NO: 66 - DNA, 52 bp, Artificial Sequence; identical to SEQ ID NO: 20
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage

SEQ ID NO: 67 - DNA, 52 bp, Artificial Sequence; identical to SEQ ID NO: 22
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage

SEQ ID NO: 68 - DNA, 52 bp, Artificial Sequence; identical to SEQ ID NO: 24
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage

SEQ ID NO: 69 - DNA, 52 bp, Artificial Sequence; identical to SEQ ID NO: 26
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage

SEQ ID NO: 70 - DNA, 52 bp, Artificial Sequence; identical to SEQ ID NO: 28
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage

SEQ ID NO: 71 - DNA, 52 bp, Artificial Sequence; identical to SEQ ID NO: 30
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage

SEQ ID NO: 72 - DNA, 52 bp, Artificial Sequence; identical to SEQ ID NO: 32
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage

SEQ ID NO: 73 - DNA, 52 bp, Artificial Sequence; identical to SEQ ID NO: 34
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage

SEQ ID NO: 74 - DNA, 52 bp, Artificial Sequence; identical to SEQ ID NO: 36
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage

SEQ ID NO: 75 - DNA, 52 bp, Artificial Sequence; identical to SEQ ID NO: 38
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage

SEQ ID NO: 76 - DNA, 52 bp, Artificial Sequence; identical to SEQ ID NO: 40
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage

SEQ ID NO: 77 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
tacactgcgcgacactgcgagcgtacaccttaatcgcgctagttagcggcgt

SEQ ID NO: 78 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
aaccgtcgagtgcaccgcgtactactaatgtcgaaccgctacgcgcactact

SEQ ID NO: 79 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
cgcggactaaggtgcgcgagtagtgttacgcgcactactaatctagccgcga

SEQ ID NO: 80 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
actagtgcgacgaactactcgcgctaaccaattcgacgcaccgatcgctagt

SEQ ID NO: 81 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
aatgtcgaaccgcgcgcgagtagtgtaccataaccgcgcaccttagtccgcg

SEQ ID NO: 82 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
gcgtcgaattggtaccgccgacttataccaatacgccgcataggtagcgcgt

SEQ ID NO: 83 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
acctagtagcgcggcgtcgaattggtactagcgcgacaacgcgtagtatggt

SEQ ID NO: 84 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
accgctcgttaccgcgcgattaaggtacgccgctaactacggtacggtcggt

SEQ ID NO: 85 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
accgccgacttatcgttcggctaggtaccaattcgacgcactgcgagcgtac

SEQ ID NO: 86 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
accttaatccgcgactgcgagcgtacacctattaccgcgcgacgcgctgtta

SEQ ID NO: 87 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
acgacgactaggtaccgctcgttacctcttgacgcgctaaccaattcgacgc

SEQ ID NO: 88 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
accatactacgcggcggttcgacattaccataaccgcgctagtgcgagcgta

SEQ ID NO: 89 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
cttgtacggcggtgcggcgtattggtaccaatacgccgctcgtcgcactagt

SEQ ID NO: 90 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
gtacgctcgcagtaccgccgacttataccttaatcgcgcactagcgcgacaa

SEQ ID NO: 91 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
acgacgactaggttatggtacggcgttagcgcgagtagtaccttagtccgcg

SEQ ID NO: 92 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
acgagcggtagtcataggtagcgcgttcttgacgcgctaaccgatcgctagt

SEQ ID NO: 93 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
accgatccgcaatgcgtcgaattggtaccataaccgcgcaccgccgtacaag

SEQ ID NO: 94 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
actagtgcgacgaactactgtcgcgaacctattaccgcgaccaatcgagcga

SEQ ID NO: 95 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
accgccgtacaagtcgcgacagtagtaaccgctcgtccgttcggcgctaggt

SEQ ID NO: 96 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
tcgtcgcactagtcgcattagtcggtagtagtacgcggtataggtagcgcgt

SEQ ID NO: 97 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
accaattcgacgctagttagcggcgtacactactcgcgcgcactcgacggtt

SEQ ID NO: 98 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
cgcggtaataggtcgcggtaataggtacgagcggtagtcacactactcgcgc

SEQ ID NO: 99 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
tcggcgagtagtttagtgcgagcgtaagtagtgcgcgtaaccaatcgagcga

SEQ ID NO: 100 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
gtcgcgcagtgtagcgcggttatggtaccataaccgcgcactagtgcgacga

SEQ ID NO: 101 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
tatgcgctcgactgcgcgattaaggtaatgtcgaaccgcagtagtacgcggt

SEQ ID NO: 102 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
actagcgcgacaacgactatgtgcgcaccaattcgacgctacgcgcactact

SEQ ID NO: 103 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
aactactcgccgacttgtacggcggtaccaattcgacgcaactaatccgcgc

SEQ ID NO: 104 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
cgcggattaaggtcttgtacggcggtacctagccgaacgtacgcgcactact

SEQ ID NO: 105 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
tagcgcgtcaagacttgtacggcggtaccgatccgcaatgcactcgacggtt

SEQ ID NO: 106 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
cgcattagtcggtgcggcgtattggtacgacgactaggtaccaatacgccgc

SEQ ID NO: 107 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
acctagtagcgcggcgcggttatggtaccgactaatgcgactagcgatcggt

SEQ ID NO: 108 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
actactcgcgctaacctagtcgtcgtaatctagccgcgatacgctcgcacta

SEQ ID NO: 109 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
accgccgctatacgcgcgattaaggtgtacgctcgcagtcgcggactaaggt

SEQ ID NO: 110 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
tacgcgcactactaaccgtcgagtgcgtacgctcgcagtaccgatcgctagt

SEQ ID NO: 111 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
gtcgcgcagtgtataacagcgcgtcgttagtgcgcgagaacgacgactaggt

SEQ ID NO: 112 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
gcgtcgaattggtcgcgtagtatggtaccgccgctatacaccaatacgccgc

SEQ ID NO: 113 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
tacgcgcactacttacgcgactaggtaccgatcgctagtcgacgcgctgtta

SEQ ID NO: 114 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
acgccgctaactatagttagcggcgtaccaattcgacgcaactaatccgcgc

SEQ ID NO: 115 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
cgcggactaaggttagttagcggcgttacgcgcactactaccgatccgcaat

SEQ ID NO: 116 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
acgacgactaggtaccgccgacttatacgccgctaactaataggtagcgcgt

SEQ ID NO: 117 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
cggatcgacggttgcgcgagtagtgtagtagtacgcggttacactgcgcgac

SEQ ID NO: 118 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
attgcggatcggtaccgccgacttataccgatccgcaattcgctcgattggt

SEQ ID NO: 119 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
actgcgagcgtacactgcgagcgtacaccttaatcgcgcaccgctcgttacc

SEQ ID NO: 120 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
actactgtcgcgatcgtcgcactagttacgctcgcactaattgcggatcggt

SEQ ID NO: 121 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
ggtaacgagcggttctcgcgcactaattagtgcgcgagaaccatactacgcg

SEQ ID NO: 122 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
actactcgcgctagcgcgattaaggtaccttaatcgcgcaactactcgccga

SEQ ID NO: 123 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
tacgcgcactactcttgtacggcggtaccaattcgacgcaaccgtcgagtgc

SEQ ID NO: 124 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
aaccgtcgatccgattgcggatcggtaccttaatcgcgcactagtgcgacga

SEQ ID NO: 125 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
agtagtgcgcgtatacactgcgcgacacactactcgcgcaccttaatccgcg

SEQ ID NO: 126 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
acgccgtaccatacgcggtaataggtagtagtgcgcgtattcggcgctaggt

SEQ ID NO: 127 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
tagcgcgtcaagaacctagcgttgcgataagtcggcggtagtagtacgcggt

SEQ ID NO: 128 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
cgcattagtcggtaatctagccgcgaaccataaccgcgcaccgatcgctagt

SEQ ID NO: 129 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
tatggtacggcgtgcggcgtattggtacgccgctaactaataagtcggcggt

SEQ ID NO: 130 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
gcgcggttatggtgcggcgtattggtacgagcggtagtcaaccgctcgtccg

SEQ ID NO: 131 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
cgactatgtgcgcaactactcgccgaaccataaccgcgctatgcgctcgact

SEQ ID NO: 132 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
tagttagcggcgtaccgctcgttaccaccttaatcgcgcaccatactacgcg

SEQ ID NO: 133 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
cgcattagtcggtagtagtgcgcgtaaaccgctcgtccgttagtgcgcgaga

SEQ ID NO: 134 - DNA, 52 bp, Artificial Sequence
  Synthetic
  misc_structure (1)..(1): 5′-phosphate
  misc_structure (1)..(2): phosphorothioate linkage
  misc_structure (1)..(1): 5′-phosphate
  misc_structure (1)..(2): phosphorothioate linkage
  misc_structure (2)..(3): phosphorothioate linkage
  misc_structure (2)..(3): phosphorothioate linkage
  misc_structure (50)..(51): phosphorothioate linkage
  misc_structure (50)..(51): phosphorothioate linkage
  misc_structure (51)..(52): phosphorothioate linkage
  misc_structure (51)..(52): phosphorothioate linkage
tagcgcgagtagtaccgactaatgcgtctcgcgcactaagactaccgctcgt

SEQ ID NO: 135 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
tacgctcgcactatcgctcgattggtaccgccgctatacaccataaccgcgc

SEQ ID NO: 136 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
accaatcgagcgaagtcgagcgcataacgcgctacctatacgccgctaacta

SEQ ID NO: 137 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
accttaatccgcgactgcgagcgtacaccgactaatgcgactactgtcgcga

SEQ ID NO: 138 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
agtagtgcgcgtatcgctcgattggttcttgacgcgctagtatagcggcggt

SEQ ID NO: 139 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
tcgtcgcactagtcggtacggtcggtgcgcacatagtcgtatggtacggcgt

SEQ ID NO: 140 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
cgcggattaaggtagtcgagcgcataaccgcgtactactacgacgactaggt

SEQ ID NO: 141 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
cgactatgtgcgctacgctcgcactaacactactcgcgcacctagcgccgaa

SEQ ID NO: 142 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
accgccgacttattctcgcgcactaatcgtcgcactagtaaccgtcgatccg

SEQ ID NO: 143 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
acctagcgttgcgaccgactaatgcgggtaacgagcggttatggtacggcgt

SEQ ID NO: 144 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
cgcgctactaggtcgcggtaataggtacctagcgttgcgacctagtcgcgta

SEQ ID NO: 145 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
cgttcggctaggtactactcgcgctacgcattagtcggttcgcgacagtagt

SEQ ID NO: 146 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
cggacgagcggttcgcggtaataggtacgacgactaggttagttagcggcgt

SEQ ID NO: 147 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
tacgctcgcactaattgcggatcggtaccgactaatgcgaccgcgtactact

SEQ ID NO: 148 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
accgaccgtaccgtatggtacggcgttcttgacgcgctaacctagcgccgaa

SEQ ID NO: 149 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
gcgcggattagttaaccgtcgagtgcacactactcgcgcactgcgagcgtac

SEQ ID NO: 150 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
accttaatccgcgaccgactaatgcgtacgcgcactactataagtcggcggt

SEQ ID NO: 151 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
accttaatccgcggcgcggttatggtaccgactaatgcgaaccgctcgtccg

SEQ ID NO: 152 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
actgcgagcgtaccttgtacggcggtacctagtagcgcgataagtcggcggt

SEQ ID NO: 153 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
ttcggcgctaggtaccttagtccgcgttcggcgctaggtacctagcgttgcg

SEQ ID NO: 154 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
acctagtcgcgtacttgtacggcggtacctagccgaacgaaccgtcgagtgc

SEQ ID NO: 155 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
accataaccgcgctacactgcgcgacaccaatacgccgctatggtacggcgt

SEQ ID NO: 156 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
acactactcgcgctacgcgactaggtaatgtcgaaccgcacgccgctaacta

SEQ ID NO: 157 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
accgactaatgcgtaacagcgcgtcgttagtgcgcgagaaccttaatcgcgc

SEQ ID NO: 158 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
acgccgtaccataaccgactaatgcgataagtcggcggtaccaatacgccgc

SEQ ID NO: 159 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
gtacgctcgcagtcgcggtaataggttcggcgagtagttaccataaccgcgc

SEQ ID NO: 160 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
cggacgagcggttgcgcggttatggtactagtgcgacgagcgcacatagtcg

SEQ ID NO: 161 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
actactcgcgctagcgcgattaaggtacgccgctaactatcgcggctagatt

SEQ ID NO: 162 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
accaattcgacgcaactaatccgcgcaccaattcgacgcagtagtgcgcgta

SEQ ID NO: 163 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
accgccgctatacacctagcgccgaagtacgctcgcagtgtatagcggcggt

SEQ ID NO: 164 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
acactactcgcgccggacgagcggttaccaatacgccgctagcgcgagtagt

SEQ ID NO: 165 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
tcgtcgcactagtaccttaatccgcgcgcaacgctaggtacactactcgcgc

SEQ ID NO: 166 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
cgcgctactaggtaccgactaatgcgcgcaacgctaggtaatgtcgaaccgc

SEQ ID NO: 167 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
acgagcggtagtcactactgtcgcgacgcaacgctaggttacactgcgcgac

SEQ ID NO: 168 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
acctagtcgcgtacgcgtagtatggtaccgatcgctagtggtaacgagcggt

SEQ ID NO: 169 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
gcggttcgacattaccgactaatgcgtatgcgctcgactacctagcgttgcg

SEQ ID NO: 170 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
actgcgagcgtactctcgcgcactaaacgccgctaactacgcgctactaggt

SEQ ID NO: 171 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
cggtacggtcggtaatctagccgcgaaccttagtccgcgaccgccgtacaag

SEQ ID NO: 172 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
tcggcgagtagttacgcgctacctattcgcggctagattacgccgctaacta

SEQ ID NO: 173 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
acgccgctaactagcgcgattaaggtgtacgctcgcagtgtcgcgcagtgta

SEQ ID NO: 174 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
agtagtgcgcgtagcggttcgacattagtagtacgcggtgcactcgacggtt

SEQ ID NO: 175 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
tcgcggctagattagtagtgcgcgtaacactactcgcgcaccttagtccgcg

SEQ ID NO: 176 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
actagcgatcggtgcgtcgaattggttagcgcgagtagttcgtcgcactagt

SEQ ID NO: 177 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
cgcggactaaggtgcgcggttatggtacactactcgcgcgcggttcgacatt

SEQ ID NO: 178 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
acgcgctacctatgcggcgtattggtataagtcggcggtaccaattcgacgc

SEQ ID NO: 179 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
accatactacgcgttgtcgcgctagtaccaattcgacgccgcgctactaggt

SEQ ID NO: 180 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
accgaccgtaccgtagttagcggcgtaccttaatcgcgcggtaacgagcggt

SEQ ID NO: 181 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
gtacgctcgcagtgcgtcgaattggtacctagccgaacgataagtcggcggt

SEQ ID NO: 182 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
taacagcgcgtcgcgcggtaataggtgtacgctcgcagtcgcggattaaggt

SEQ ID NO: 183 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
gcgtcgaattggttagcgcgtcaagaggtaacgagcggtacctagtcgtcgt

SEQ ID NO: 184 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
tacgctcgcactagcgcggttatggtaatgtcgaaccgccgcgtagtatggt

SEQ ID NO: 185 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
actagtgcgacgagcggcgtattggtaccaatacgccgcaccgccgtacaag

SEQ ID NO: 186 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
ttgtcgcgctagtgcgcgattaaggtataagtcggcggtactgcgagcgtac

SEQ ID NO: 187 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
cgcggactaaggtactactcgcgctaacgccgtaccataacctagtcgtcgt

SEQ ID NO: 188 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
actagcgatcggttagcgcgtcaagaacgcgctacctatgactaccgctcgt

SEQ ID NO: 189 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
cttgtacggcggtgcgcggttatggtaccaattcgacgcattgcggatcggt

SEQ ID NO: 190 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
tcgctcgattggtcgcggtaataggttcgcgacagtagttcgtcgcactagt

SEQ ID NO: 191 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
acctagcgccgaacggacgagcggttactactgtcgcgacttgtacggcggt

SEQ ID NO: 192 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
acgcgctacctataccgcgtactactaccgactaatgcgactagtgcgacga

SEQ ID NO: 193 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
aaccgtcgagtgcgcgcgagtagtgtacgccgctaactagcgtcgaattggt

SEQ ID NO: 194 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
gcgcgagtagtgtgactaccgctcgtacctattaccgcgacctattaccgcg

SEQ ID NO: 195 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
tcgctcgattggttacgcgcactacttacgctcgcactaaactactcgccga

SEQ ID NO: 196 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
tcgtcgcactagtgcgcggttatggtaccataaccgcgctacactgcgcgac

SEQ ID NO: 197 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
accgcgtactactgcggttcgacattaccttaatcgcgcagtcgagcgcata

SEQ ID NO: 198 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
agtagtgcgcgtagcgtcgaattggtgcgcacatagtcgttgtcgcgctagt

SEQ ID NO: 199 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
gcgcggattagttgcgtcgaattggtaccgccgtacaagtcggcgagtagtt

SEQ ID NO: 200 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
agtagtgcgcgtacgttcggctaggtaccgccgtacaagaccttaatccgcg

SEQ ID NO: 201 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
aaccgtcgagtgcattgcggatcggtaccgccgtacaagtcttgacgcgcta

SEQ ID NO: 202 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
gcggcgtattggtacctagtcgtcgtaccaatacgccgcaccgactaatgcg

SEQ ID NO: 203 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
accgatcgctagtcgcattagtcggtaccataaccgcgccgcgctactaggt

SEQ ID NO: 204 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
tagtgcgagcgtatcgcggctagattacgacgactaggttagcgcgagtagt

SEQ ID NO: 205 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
accttagtccgcgactgcgagcgtacaccttaatcgcgcgtatagcggcggt

SEQ ID NO: 206 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
actagcgatcggtactgcgagcgtacgcactcgacggttagtagtgcgcgta

SEQ ID NO: 207 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
acctagtcgtcgttctcgcgcactaacgacgcgctgttatacactgcgcgac

SEQ ID NO: 208 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
gcggcgtattggtgtatagcggcggtaccatactacgcgaccaattcgacgc

SEQ ID NO: 209 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
taacagcgcgtcgactagcgatcggtacctagtcgcgtaagtagtgcgcgta

SEQ ID NO: 210 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
gcgcggattagttgcgtcgaattggtacgccgctaactatagttagcggcgt

SEQ ID NO: 211 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
attgcggatcggtagtagtgcgcgtaacgccgctaactaaccttagtccgcg

SEQ ID NO: 212 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
acgcgctacctattagttagcggcgtataagtcggcggtacctagtcgtcgt

SEQ ID NO: 213 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
gtcgcgcagtgtaaccgcgtactactacactactcgcgcaaccgtcgatccg

SEQ ID NO: 214 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
accaatcgagcgaattgcggatcggtataagtcggcggtaccgatccgcaat

SEQ ID NO: 215 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
ggtaacgagcggtgcgcgattaaggtgtacgctcgcagtgtacgctcgcagt

SEQ ID NO: 216 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
accgatccgcaattagtgcgagcgtaactagtgcgacgatcgcgacagtagt

SEQ ID NO: 217 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
cgcgtagtatggttctcgcgcactaattagtgcgcgagaaccgctcgttacc

SEQ ID NO: 218 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
tcggcgagtagttgcgcgattaaggtaccttaatcgcgctagcgcgagtagt

SEQ ID NO: 219 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
gcactcgacggttgcgtcgaattggtaccgccgtacaagagtagtgcgcgta

SEQ ID NO: 220 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
tcgtcgcactagtgcgcgattaaggtaccgatccgcaatcggatcgacggtt

SEQ ID NO: 221 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
cgcggattaaggtgcgcgagtagtgtgtcgcgcagtgtatacgcgcactact

SEQ ID NO: 222 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
acctagcgccgaatacgcgcactactacctattaccgcgtatggtacggcgt

SEQ ID NO: 223 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
accgcgtactactaccgccgacttatcgcaacgctaggttcttgacgcgcta

SEQ ID NO: 224 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
actagcgatcggtgcgcggttatggttcgcggctagattaccgactaatgcg

SEQ ID NO: 225 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
accgccgacttattagttagcggcgtaccaatacgccgcacgccgtaccata

SEQ ID NO: 226 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
cggacgagcggttgactaccgctcgtaccaatacgccgcaccataaccgcgc

SEQ ID NO: 227 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
agtcgagcgcatagcgcggttatggttcggcgagtagttgcgcacatagtcg

SEQ ID NO: 228 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
cgcgtagtatggtgcgcgattaaggtggtaacgagcggtacgccgctaacta

SEQ ID NO: 229 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
tctcgcgcactaacggacgagcggtttacgcgcactactaccgactaatgcg

SEQ ID NO: 230 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
acgagcggtagtcttagtgcgcgagacgcattagtcggtactactcgcgcta

SEQ ID NO: 231 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
gcgcggttatggtgtatagcggcggtaccaatcgagcgatagtgcgagcgta

SEQ ID NO: 232 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
tagttagcggcgtataggtagcgcgttatgcgctcgacttcgctcgattggt

SEQ ID NO: 233 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
tcgcgacagtagtcgcattagtcggtgtacgctcgcagtcgcggattaaggt

SEQ ID NO: 234 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
accgccgctatactagcgcgtcaagaaccaatcgagcgatacgcgcactact

SEQ ID NO: 235 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
acgccgtaccatacgactatgtgcgcaccgaccgtaccgactagtgcgacga

SEQ ID NO: 236 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
acctagtcgtcgtagtagtacgcggttatgcgctcgactaccttaatccgcg

SEQ ID NO: 237 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
ttcggcgctaggtgcgcgagtagtgttagtgcgagcgtagcgcacatagtcg

SEQ ID NO: 238 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
cggatcgacggttactagtgcgacgattagtgcgcgagaataagtcggcggt

SEQ ID NO: 239 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
acgccgtaccataaccgctcgttacccgcattagtcggtcgcaacgctaggt

SEQ ID NO: 240 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
tacgcgactaggtcgcaacgctaggtacctattaccgcgacctagtagcgcg

SEQ ID NO: 241 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
actactgtcgcgaaccgactaatgcgtagcgcgagtagtacctagccgaacg

SEQ ID NO: 242 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
acgccgctaactaacctagtcgtcgtacctattaccgcgaaccgctcgtccg

SEQ ID NO: 243 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
agtagtacgcggtcgcattagtcggtaccgatccgcaattagtgcgagcgta

SEQ ID NO: 244 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
ttcggcgctaggttagcgcgtcaagaacgccgtaccatacggtacggtcggt

SEQ ID NO: 245 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
gtacgctcgcagtgcgcgagtagtgtgcactcgacggttaactaatccgcgc

SEQ ID NO: 246 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
accgccgacttatagtagtgcgcgtacgcattagtcggtcgcggattaaggt

SEQ ID NO: 247 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
cggacgagcggttcgcattagtcggtaccataaccgcgccgcggattaaggt

SEQ ID NO: 248 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
accgccgacttatcgcgctactaggtaccgccgtacaaggtacgctcgcagt

SEQ ID NO: 249 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
cgcaacgctaggtacctagcgccgaacgcggactaaggtacctagcgccgaa

SEQ ID NO: 250 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
gcactcgacggttcgttcggctaggtaccgccgtacaagtacgcgactaggt

SEQ ID NO: 251 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
acgccgtaccatagcggcgtattggtgtcgcgcagtgtagcgcggttatggt

SEQ ID NO: 252 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
tagttagcggcgtgcggttcgacattacctagtcgcgtagcgcgagtagtgt

SEQ ID NO: 253 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
gcgcgattaaggttctcgcgcactaacgacgcgctgttacgcattagtcggt

SEQ ID NO: 254 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
gcggcgtattggtaccgccgacttatcgcattagtcggttatggtacggcgt

SEQ ID NO: 255 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
gcgcggttatggtaactactcgccgaacctattaccgcgactgcgagcgtac

SEQ ID NO: 256 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
cgactatgtgcgctcgtcgcactagtaccataaccgcgcaaccgctcgtccg

SEQ ID NO: 257 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
aatctagccgcgatagttagcggcgtaccttaatcgcgctagcgcgagtagt

SEQ ID NO: 258 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
tacgcgcactactgcgtcgaattggtgcgcggattagttgcgtcgaattggt

SEQ ID NO: 259 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
accgccgctatacactgcgagcgtacttcggcgctaggtgtatagcggcggt

SEQ ID NO: 260 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
actactcgcgctagcggcgtattggtaaccgctcgtccggcgcgagtagtgt

SEQ ID NO: 261 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
gcgcgagtagtgtacctagcgttgcgcgcggattaaggtactagtgcgacga

SEQ ID NO: 262 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
gcggttcgacattacctagcgttgcgcgcattagtcggtacctagtagcgcg

SEQ ID NO: 263 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
gtcgcgcagtgtaacctagcgttgcgtcgcgacagtagtgactaccgctcgt

SEQ ID NO: 264 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
accgctcgttaccactagcgatcggtaccatactacgcgtacgcgactaggt

SEQ ID NO: 265 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
cgcaacgctaggtagtcgagcgcatacgcattagtcggtaatgtcgaaccgc

SEQ ID NO: 266 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
acctagtagcgcgtagttagcggcgtttagtgcgcgagagtacgctcgcagt

SEQ ID NO: 267 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
cttgtacggcggtcgcggactaaggttcgcggctagattaccgaccgtaccg

SEQ ID NO: 268 - DNA, 52 bp, Artificial Sequence
  Synthetic
  misc_structure (1)..(1): 5′-phosphate
  misc_structure (1)..(2): phosphorothioate linkage
  misc_structure (2)..(3): phosphorothioate linkage
  misc_structure (50)..(51): phosphorothioate linkage
  misc_structure (51)..(52): phosphorothioate linkage
tagttagcggcgtaatctagccgcgaataggtagcgcgtaactactcgccga

SEQ ID NO: 269 - DNA, 24 bp, Artificial Sequence
  Synthetic
acgcgactatacgcgcaatatggt

SEQ ID NO: 270 - DNA, 25 bp, Artificial Sequence
  Synthetic
ctagcgatactacgcgatacgagat

SEQ ID NO: 271 - DNA, 26 bp, Artificial Sequence
  Synthetic
catagcggtattacgcgagattacga

SEQ ID NO: 272 - DNA, 21 bp, Artificial Sequence
  Synthetic
cgcgagtacgtacgattaccg

SEQ ID NO: 273 - DNA, 21 bp, Artificial Sequence
  Synthetic
acgcgcgactatacgcgcctc

(19) United States
(12) Patent Application Publication
(10) Pub. No.: US 2022/0025365 A1
(43) Pub. Date: Jan. 27, 2022

(54) METHODS FOR NOMINATION OF NUCLEASE ON-/OFF-TARGET EDITING LOCATIONS, DESIGNATED "CTL-seq" (CRISPR Tag Linear-seq)

(71) Applicant: INTEGRATED DNA TECHNOLOGIES, INC., Coralville, IA (US)

(72) Inventors: Matthew MCNEILL, Iowa City, IA (US); Rolf TURK, Iowa City, IA (US); Garrett RETTIG, Coralville, IA (US); Ellen BLACK, Swisher, IA (US); Yongming SUN, San Ramon, CA (US); Chris SAILOR, Cedar Rapids, IA (US); Yu WANG, North Grafton, MA (US); Keith GUNDERSON, Iowa City, IA (US); Kyle KINNEY, Iowa City, IA (US)

(21) Appl. No.: 17/382,945

(22) Filed: Jul. 22, 2021

Related U.S. Application Data
(60) Provisional application No. 63/055,460, filed on Jul. 23, 2020.

(51) Int. Cl. C12N 15/11; C12N 9/22; C12Q 1/6853

(52) U.S. Cl. CPC C12N 15/111; C12N 2310/20; C12Q 1/6853; C12N 9/22

(57) ABSTRACT

Described herein are methods for identifying and nominating on- and off-target CRISPR editing sites with improved accuracy and sensitivity.

### CROSS REFERENCE TO RELATED APPLICATIONS

This application claims priority to U.S. Provisional Patent Application No. 63/055,460, filed on Jul. 23, 2020, which is incorporated by reference herein in its entirety.

### REFERENCE TO SEQUENCE LISTING

This application is filed with a Computer Readable Form of a Sequence Listing in accordance with 37 C.F.R. § 1.821(c). The text file submitted by EFS, “013670-9056-US02_sequence_listing_19-JUL-2021_ST25.txt” contains 273 sequences, was created on Jul. 19, 2021, has a file size of 153 Kbytes, and is hereby incorporated by reference in its entirety.

### TECHNICAL FIELD

Described herein are methods for identifying and nominating on- and off-target CRISPR editing sites with improved accuracy and sensitivity.

### BACKGROUND

CRISPR (clustered regularly interspaced short palindromic repeats) has revolutionized genomics by permitting the simple introduction of changes to the genetic code. CRISPR systems, such as Cas9 and Cas12a proteins, are guided to their target by RNA oligonucleotide sequences bound by the Cas proteins (forming ribonucleoprotein protein; RNP), where the enzyme creates double stranded breaks (DSBs) in DNA sequences. Native cellular machinery repairs DSBs, generally using non-homologous end joining (NHEJ) or homology directed repair (HDR) molecular pathways. DNA repaired through NHEJ, which occurs at on- and off-target locations, often contains indels (insertions/deletions), which can lead to mutations and change the function of encoded genes. Thus, identifying these locations is critical to deconvoluting the impact of on- and off-target editing on biological phenotypes.

To date, no “gold standard” method exists to identify or nominate off-target editing locations for CRISPR or other nucleases. Many methods have been developed. These methods use a variety of strategies, including the detection of endogenous repair machinery assembled at DSBs (Discover-Seq [1]), the integration of a DNA tag sequence into the host cell genome (GUIDE-Seq; see U.S. Pat. No. 9,822,407), iGUIDE [2, 3]), or by cutting DNA in vitro (BLISS [4], CIRCLE-Seq [5], SiteSeq [6]).

Cellular or cell based (sometimes referred to as in vivo) and biochemical (sometimes referred to as in vitro) off-target assay nomination systems each have their advantages. Proteins bound to the DNA and epigenetic marks modify the function of nuclease activity, suggesting that cellular or cell based methods may better identify actual editing targets [7]. However, biochemical methods have nominated sites not identified through cellular or cell based methods, suggesting biochemical methods may be more comprehensive [5, 6]. Nevertheless, these current tools tend to have imperfect sensitivity [5, 6] (see FIG. 1).

What is needed is a method for detecting and nominating on- and off-target CRISPR editing sites with improved accuracy and sensitivity.

### SUMMARY

One embodiment described herein is a method for identifying and nominating on- and off-target CRISPR edited sites with improved accuracy and sensitivity, the process comprising the steps of: (a) co-delivering a guide sequence RNA (sgRNA) or a two-part CRISPR RNA:trans-activating crRNA (crRNA:tracrRNA) duplex, one or more tag sequences, and an RNA-guided endonuclease to cells; (b) incubating the cells for a period of time sufficient for double strand breaks to occur; (c) isolating genomic DNA from the cells, fragmenting the genomic DNA, and ligating the fragmented genomic DNA to a unique molecular index containing a universal adapter sequence; (d) amplifying the ligated DNA fragments using primers targeting the tag and universal adapter sequences to produce a first set of amplified sequences; (e) amplifying the first set of amplified sequences using universal sequencing primers targeting the tails of Tag-pTOP or Tag-pBOT primers to produce a second set of amplified sequences; (f) sequencing the pooled sequences and obtaining sequencing data; and (g) identifying on-/off-target CRISPR editing loci. In one aspect, the universal sequencing primers target SP1 or SP2 sequence (SEQ ID NO: 7, 8) tails on the Tag-pTOP or Tag-pBOT primers to produce a second set of amplified sequences. In another aspect, the universal sequencing primers target predesigned non-homologous sequence (SEQ ID NO: 269-273) tails on the Tag-pTOP or Tag-pBot primers to produce a second set of amplified sequences. In another aspect, the universal sequencing primers target predesigned 13-mer tails on the Tag-pTOP or Tag-pBot primers to produce a second set of amplified sequences. In another aspect, step (g) comprises executing on a processor: (i) aligning the sequence data to a reference genome; (ii) identifying on-/off-target CRISPR editing loci; and (iii) outputting the alignment, analysis, and results data as custom-formatted files, tables or graphics. In another aspect, the method further comprises a step following step (e) comprising: (e1) normalizing the second set of amplified sequences to produce concentration normalized libraries, pooling the normalized libraries with other samples to produce pooled libraries; and continuing with steps (f)-(i). In another aspect, step (d) uses a suppression PCR method. In another aspect, the RNA-guided endonuclease comprises an endogenously-expressed Cas enzyme, a Cas expression vector, a Cas protein, or a Cas RNP complex. In another aspect, the RNA-guided endonuclease comprises an endogenously-expressed Cas9 enzyme, a Cas9 expression vector, a Cas9 protein, or a Cas9 RNP complex. In another aspect, the cells comprise human or mouse cells. In another aspect, the period of time is about 24 hours to about 96 hours. In another aspect, multiple tag sequences are co-delivered. In another aspect, the tag sequences comprise double-stranded deoxyribooligonucleotides (dsDNA) comprising 52-base pairs. In another aspect, the tag sequences comprise a 5′-terminal phosphate, and phosphorothioate linkages between the 1^{st} and 2^{nd}, 2^{nd} and 3^{rd}, 50^{th} and 51^{st}, and 51^{st} and 52^{nd} nucleotides. In another aspect, the tag sequences comprise a double stranded DNA comprising the complementary top and bottom strand pairs of SEQ ID NO: 1-2 or 7-268.

Other embodiments described herein are on- and off-target CRISPR editing sites identified or nominated using the methods described herein.

Another embodiment described herein is a method for designing 52-base pair tag sequences, the method comprising, executing on a processor: (a) randomly generating 13-nucleotide sequences with 40-90% GC content, max homopolymer length A:2, C:3, G:2, T:2, weighted homopolymer rate <20, self-folding Tₘ<50° C., and self-dimer Tₘ<50° C.; (b) removing sequences that perfectly align to a particular genome or that are homopolymers or GG or CC dinucleotide motifs and obtaining a set of 13-mers; (c) selecting a subset of the 13-mer sequences that contain one or less CC or GG dinucleotide motifs; (d) concatenating four of the of 13-mer subset sequences to form random 52-mer sequences; (e) aligning the random 52-mer sequences to a genome; (f) removing the random 52-mer sequences that have similarity to the genome to produce a subset of 52-mer sequences; and (h) outputting the subset of 52-mer sequences and generating the complementary strands to produce double stranded 52-base pair tag sequences. In one aspect, the genome is human or mouse. In another aspect, the 52-base pair tag sequences are-non complementary to the genome. In another aspect, the method further comprises designing primers for the 52-base pair tag sequences. In another aspect, the 52-base pair tag sequences comprise a 5′-terminal phosphate, and phosphorothioate linkages between the 1^{st} and 2^{nd}, 2^{nd} and 3^{rd}, 50^{th} and 51^{st}, and 51^{st} and 52^{nd} nucleotides of the 52-base pair tag sequences. In another aspect, the method further comprises synthesizing oligonucleotides comprising the 52-base pair tag sequences, the complement of the 52-base pair tag sequences, or primers for the 52-base pair tag sequences.

Other embodiments described herein are one or more 52-base pair tag sequences designed using the methods described herein. In one aspect, the 52-base pair tag sequence comprises a double stranded DNA comprising the top and bottom strand pairs of SEQ ID NO: 1-2 or 7-268.

Another embodiment described herein is a method for designing primers partially complementary to the 52-base pair tag sequences of claim 23 and an adapter primer, the method comprising, executing on a processor: (a) designing tag primers that are partially complementary to the top and bottom strands of tag sequences; and (b) designing an adapter primer that is partially complementary to the top strand of the adapter sequence; wherein: the tag primers comprise a 5′-universal tail sequence; and the adapter primer comprises a sequence complementary to the tails of Tag-pTOP or Tag-pBOT primers. In one aspect, the 5′-universal tail sequence is complementary to an SP1 or SP2 sequence (SEQ ID NO: 7, 8), a locus specific segment, a ribonucleotide (rN) 6-nucleotides from the 3′-end, a 3′-end mismatch, a 3′-end block (3′-C₃ spacer), a predesigned non-homologous sequence (SEQ ID NO: 269-273), or a predesigned 13-mer sequence. In another aspect, the primers partially complementary to top and bottom strands of the tag sequences comprise a tail sequence complementary to the SP1 sequence (SEQ ID NO: 7) and the adapter primer comprises a sequence complementary to the SP2 sequence (SEQ ID NO: 8) tail on the Tag-pTOP or Tag-pBOT primers; or the primers partially complementary to top and bottom strands of the tag sequences comprise a tail sequence complementary to the SP2 sequence (SEQ ID NO: 8) and the adapter primer comprises a sequence complementary to the SP1 sequence (SEQ ID NO: 7) tail on the Tag-pTOP or Tag-pBOT primers. In another aspect, the amplification of a nucleic acid molecule with the primers that are complementary to the top and bottom strands of tag sequences and primers that are complementary to the top strand of the adapter sequence produces a PCR product that comprises a portion of the tag sequence, a sgDNA sequence, and the adapter sequence. In another aspect, the method further comprises synthesizing oligonucleotides comprising the sequences of the forward and reverse tag primers and the adapter primer. In another aspect, the 52-base pair tag sequences and primers partially complementary to the 52-base pair tag sequences are designed and selected using an algorithm predicting whether the primers are likely to be partially complementary and have a propensity to form primer-dimers.

Other embodiments described herein are one or more primers partially complementary to the 52-base pair tag sequences and one or more adapter primers designed using the methods described herein. In one aspect, the primers comprise the sequences of SEQ ID NO: 3, 4; and the adapter primer, wherein the adapter primer comprises the sequence of SEQ ID NO: 5.

Another embodiment described herein is the use of one or more double-stranded 52-base pair tag sequences for identifying on- and off-target CRISPR editing sites.

### DESCRIPTION OF THE DRAWINGS

FIG. 1 shows fraction of reads shared by three biological replicates are shown in white sectors; whereas reads shared by two replicates, or present in a single replicate, are shown in black sectors. Table 1 shows GUIDE-seq [3] based nomination for 4 different gRNAs in triplicate in a 96-well format. gRNA complexes were generated by mixing equimolar amounts of Alt-R crRNA-XT and Alt-R tracrRNA. HEK293 cells stably expressing Cas9 were transfected with 10 μM gRNA and 0.5 μM dsODN GUIDE-seq tag using the Nucleofector™ system (Lonza). After 72 hrs, genomic DNA (gDNA) was isolated. Genomic DNA was fragmented, and adapters were ligated using the Lotus DNA library preparation kit (IDT). Libraries were generated by amplification from the inserted tag to the ligated adapters [3]. Libraries were then sequenced in paired-end fashion on an IIlumina® platform.

FIG. 2 shows that GUIDE-Seq finds more off-target locations than can be validated through rhAmpSeq targeted amplification. Presented results are an aggregate of 331 GUIDE-Seq nominated sites when delivering gRNA sequences (internally named: AR, CTNNB1, EMX1, GRHPR, HPRT38087, HPRT38285, VEGFA) into HEK293 cells stably expressing WT Cas9. GUIDE-seq nominated off-targets assigned 0.1% of the total reference genome aligned reads for each guide were designed and targeted by one rhAmpSeq panel all reference genome aligned. In subsequent experiments, gRNAs were again delivered to the same cells, and editing was assayed with rhAmpSeq. Targets were called “edited” if the treated condition had observed indels ≥the untreated control sample at %.

FIG. 3 illustrates that GUIDE-Seq tag integration rate varies. The graph shows the percentage of Tag integration (normalized to % Editing) for 118 unique Cas9 on/off-target sites that had InDel editing in rhAmpSeq panels targeting GUIDE-Seq nominated on/off-target loci for guide sequences targeting the RAG1, RAG2, and EMX1 genes. Each guide was co-delivered with the 34-base pair GUIDE-Seq, dsODN tag into HEK293 cells stably expressing Cas9 by nucleofection. DNA was extracted 72 hrs later, amplified by rhAmpSeq multiplex PCR, sequenced on an Illumina® MiSeq, and analyzed through a custom pipeline. The normalized tag integration rate is calculated as the percentage of sequenced reads at each target containing the tag sequence divided by the total reads containing an allele divergent from the reference genome (indicating Cas9 editing).

FIG. 4 shows the design of rhAmpSeq primers against alien sequence tags. A cartoon diagram shows the steps of the design process using the rhAmpSeq design pipeline including design of forward primers against the top (1) and bottom (2) strands, discarding unneeded primers, and selecting tag-targeting primers that have 5′-overlapping, but not 3′-overlapping sequences, so that the top/bottom strand primer dimers would hairpin (3).

FIG. 5 shows an overview of the rhAmpSeq design pipeline used to construct the overlapping primer designs. In the pipeline, a known sequence is appended onto the 5′-end and 3′-end of each tag sequence, the inputs are quality-controlled and assays (shown in FIG. 4A) are designed against the top and bottom strand of each tag. Primers targeting each tag strand are paired such that at least 4-nucleotides 3′ of the RNA nucleotide do not overlap between primers targeting the same tag, and primer pairs are ranked and selected. Hg38 and mm38 acronyms represent versions of the human and mouse genomes, respectively.

FIG. 6 illustrates hairpin formation if overlapping primers generate PCR amplicons. The diagram shows a representative target sequence and hairpin PCR product of undesired short amplicons from overlapping primer regions with complementary 5′ primer tail ends at the 3′- and 5′-end of the PCR product.

FIG. 7 shows the number of target sites (black bars) with integration of the specified single tag (SEQ ID NO: 9-40) or pools of tags described in Table 5 (SEQ ID NO: 9-40, 45-268). The striped bar (CTLmax) shows the maximum number of target sites that theoretically can be found if a combination of the single tags (SEQ ID NO: 9-40) is used (23 sites out of a maximum of 32 sites). Pool A1 contains all the single tags (SEQ ID NO: 9-40). Pools B1-6 contain 16 different tags each (SEQ ID NO: 45-268). Pool C1 contains all tags tested (SEQ ID NO: 9-40, 45-268). Integration events were determined using an in-house data analysis tool.

FIG. 8 shows the number of target sites (black bars) with integration of the specified single tag (SEQ ID NO: 9-40) or pools of tags described in Table 5 (SEQ ID NO: 9-40, 45-268). The striped bar (CTLmax) shows the maximum number of target sites that theoretically can be found if a combination of the single tags (SEQ ID NO: 9-40) is used (47 sites out of a maximum of 53 sites). Pool A1 contains all the single tags (SEQ ID NO: 9-40). Pools B1-6 contain 16 different tags each (SEQ ID NO: 45-268). Pool C1 contains all tags tested (SEQ ID NO: 9-40, 45-268). Integration events were determined using an in-house data analysis tool.

### DETAILED DESCRIPTION

Described herein are methods for detecting and nominating on- and off-target CRISPR editing sites with improved accuracy and sensitivity. The intracellular context information is maintained by building upon prior in vivo nomination methods. The sensitivity is expanded by co-delivering a set of unique, predefined sequence tags. In one aspect, the co-delivered set of predefined unique tags may range from 13-80 base pairs. In another aspect, the co-delivered set of predefined tags may be comprised of 13 base pair tag sequence tags, 26 base pair tag sequence tags, 39 base pair tag sequence tags, 52 base pair tag sequence tags, 65 base pair tag sequence tags, or 78 base pair tag sequence tags. In another aspect, the unique predefined tags are a set of 52-base pair tag sequence tags (the increased length of the sequence tags improves the ability to find good primer landing sites for rhPrimers). This limitation is believed to be mitigated by using a diversity of tag sequences that are distinct from human and mouse genomes. The specificity is improved by building upon Integrated DNA Technologies (IDT)'s rhAmp technology that uses RNAaseH2 (Pyrococcus abyssi) to unblock primers that have correctly annealed to their target; this yields lower rates of false priming. Specificity can be further enhanced by only nominating targets using reads that contain an expected tag sequence at the 5′-end. The incorporation of suppression PCR into this method permits ease of use. The prior in vivo methods (e.g., GUIDE-seq and iGUIDE) require parallel PCR reactions (2 pool amplification) to amplify by annealing to and extending from the top and bottom strand of the tags. Here, suppression PCR is used to allow both pools to be amplified simultaneously without causing problematic dimer sequences.

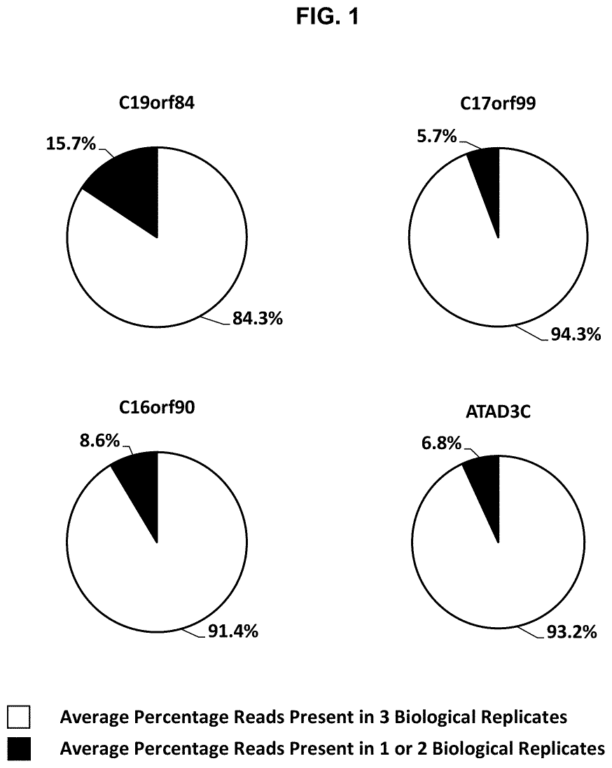

A GUIDE-Seq dsDNA tag was co-delivered with one guide RNA to HEK293 cells constitutively expressing Cas9 using nucleofection. See U.S. Pat. No. 9,822,407, which is incorporated by reference herein for such teachings. A total of four different guide RNAs were tested in this fashion. Ribonucleoprotein complexes (RNPs) between the expressed Cas9 and guide RNA form within the cells, introducing double stranded breaks. Repaired breaks can contain the co-delivered tags. After delivery, cells were incubated, and the resulting DNA was extracted. Target amplification was performed according to the GUIDE-Seq protocol and assayed with a modified version of the GUIDE-Seq analytical pipeline (github.com/aryeelab/guideseq). Nominated targets were compared between three biological replicates (unique guideRNA+Tag co-deliveries). Not all nominated targets were common to all biological replicates (commonly/total nominated targets: 7/31, 6/19, 2/4, 3/5 respectively; see Table 1). However, >90% of the total reads, attributed to any target, were attributed to common targets (on average; see FIG. 1).

TABLE 1
Identified off-target sites for four different gRNAs and relative
level of editing at off-target sites compared to the on-target site

| Location | C19orf84_BR1 | C19orf84_BR2 | C19orf84_BR3 |
|---|---|---|---|
| chr19_51389306 | 100.00% | 100.00% | 100.00% |
| chr9_20224748 | 38.55% | 16.43% | 29.00% |
| chr4_28036434 | 16.33% | 13.05% | 14.36% |
| chr15_74256506 | 14.30% | 18.18% | 25.17% |
| chr2_171312919 | 11.40% | 8.51% | 7.93% |
| chr8_65742269 | 10.82% | 1.17% | 10.40% |
| chr13_96554656 | 8.70% | 0.00% | 0.00% |
| chr4_86807920 | 8.50% | 9.21% | 1.92% |
| chr3_124485356 | 6.57% | 0.00% | 0.00% |
| chr9_20330398 | 5.60% | 0.00% | 0.00% |
| chr11_71298123 | 5.12% | 0.00% | 0.00% |
| chr7_101729696 | 4.83% | 0.00% | 9.58% |
| chr19_10923882 | 3.67% | 3.03% | 0.00% |
| chr10_15548456 | 3.57% | 15.38% | 0.00% |
| chr12_117097457 | 2.80% | 0.00% | 2.60% |
| chr22_33493900 | 2.13% | 0.00% | 4.79% |
| chrX_149763439 | 2.13% | 0.00% | 3.83% |
| chr17_7435217 | 1.93% | 0.00% | 0.55% |
| chr12_26286721 | 1.74% | 0.00% | 5.06% |
| chr16_49704848 | 1.26% | 5.01% | 7.11% |
| chr12_51288216 | 1.06% | 0.00% | 0.00% |
| chr12_56010621 | 0.87% | 0.00% | 0.00% |
| chr13_29717148 | 0.48% | 0.00% | 0.00% |
| chr1_3088065 | 0.29% | 0.00% | 0.00% |
| chr15_73442915 | 0.19% | 0.00% | 0.55% |
| chr10_118045968 | 0.19% | 0.00% | 0.00% |
| chr14_102199972 | 0.00% | 0.00% | 0.68% |
| chr18_56334679 | 0.00% | 0.00% | 2.33% |
| chr21_36426137 | 0.00% | 0.00% | 2.19% |
| chr5_139002763 | 0.00% | 0.00% | 3.83% |
| chrX_58291642 | 0.00% | 0.00% | 3.83% |
| Location | C17orf99_BR1 | C17orf99_BR2 | C17orf99_BR3 |
| chr17_78164110 | 100.00% | 100.00% | 100.00% |
| chr22_24471716 | 15.00% | 13.24% | 10.86% |
| chr10_101156881 | 6.22% | 11.07% | 9.79% |
| chr3_170476431 | 5.86% | 3.97% | 4.57% |
| chr17_17692965 | 4.94% | 0.66% | 8.62% |
| chr15_73400031 | 3.93% | 4.63% | 5.73% |
| chr19_15238775 | 0.00% | 0.00% | 2.56% |
| chr2_18362316 | 0.00% | 0.00% | 1.59% |
| chr2_171087784 | 0.00% | 0.54% | 0.84% |
| chr22_19959968 | 0.00% | 1.26% | 0.19% |
| chr22_32114104 | 0.00% | 0.00% | 4.06% |
| chr4_129034015 | 0.00% | 0.00% | 0.33% |
| chr5_61219030 | 0.00% | 0.00% | 0.33% |
| chr5_66209615 | 0.00% | 0.00% | 1.86% |
| chr7_69709389 | 0.00% | 0.12% | 2.75% |
| chr7_158662844 | 0.00% | 1.44% | 5.27% |
| chrX_9567397 | 0.00% | 0.00% | 0.23% |
| chr19_55657073 | 0.00% | 0.66% | 0.00% |
| chr22_43788032 | 0.00% | 2.47% | 0.00% |
| Location | C16orf90_BR1 | C16orf90_BR2 | C16orf90_BR3 |
| chr16_3494817 | 100.00% | 100.00% | 100.00% |
| chr2_109189307 | 75.32% | 4.27% | 52.05% |
| chr22_24586001 | 45.45% | 0.00% | 0.00% |
| chr10_104736568 | 0.00% | 0.00% | 8.22% |
| Location | ATAD3C_BR1 | ATAD3C_BR2 | ATAD3C_BR3 |
| chr1_1450685 | 100.00% | 100.00% | 100.00% |
| chr1_1503588 | 11.73% | 10.07% | 9.27% |
| chr1_1516015 | 2.47% | 1.86% | 5.14% |
| chr19_32167960 | 26.34% | 0.93% | 0.00% |
| chr2_111077960 | 0.00% | 1.12% | 0.00% |

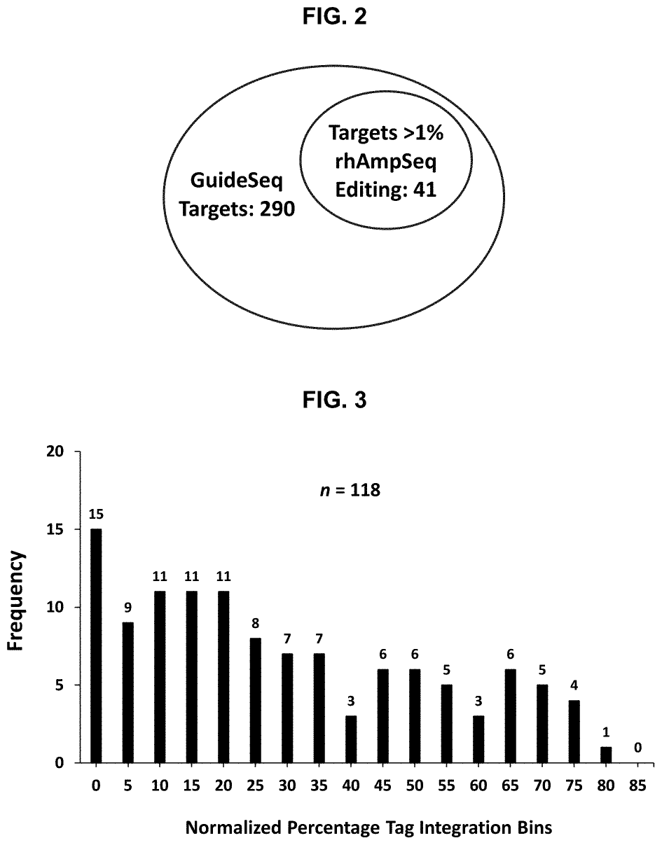

Additionally, nominated targets may not be replicable or detectable using orthogonal methods. Using the GUIDE-Seq method, the GUIDE-Seq DNA tag was co-delivered with each of 6 guides (each tag is delivered with one guide RNA) to HEK293 cells constitutively expressing Cas9 using nucleofection. rhAmpSeq multiplex amplicon panels were designed to amplify the nominated targets, and we quantified editing in biological replicates. Of the 331 targets nominated by GUIDE-Seq, only 41 (12%) could be verified with rhAmpSeq (see FIG. 2).

dsDNA tag sequences co-delivered with the guide RNAs into a stably expressing CRISPR cell line, which are used in the NHEJ repair, are incorporated at varying rates. Here, the GUIDE-Seq dsDNA tag was co-delivered with each of 6 guides into HEK293 cells constitutively expressing Cas9. In another aspect, the dsDNA tag sequences co-delivered with CRISPR RNP, which are used in the NHEJ repair, are incorporated at varying rates. Here, the GUIDE-Seq dsDNA tag was co-delivered with each of 6 guides into HEK293 cells constitutively expressing Cas9. rhAmpSeq panels were developed to amplify nominated targets, and in biological replicates, the rates of tag integration were analyzed using a custom analytical pipeline. These results demonstrate that tags are incorporated at 0-85% of edited genomic copies, varying by target (see FIG. 3). Without being bound by any theory, it is hypothesized that the rate varies by sequence context.

Described herein are methods to improve the signal to noise ratio by combining Integrated DNA Technology's rhAmpSeg™ technology, suppression PCR, and novel alien DNA sequence designs to nominate nuclease off-target editing locations within a host genome.

In this method, Cas9, a sgRNA or a two-part CRISPR RNA:trans-activating crRNA (crRNA:tracrRNA) duplex, and one or more double stranded DNA (dsDNA) tag sequences are delivered to cells. Co-delivering multiple tags permits improved tag integration at off-target sites (see below). The tag sequences have sequence content significantly different (i.e., alien) to the host genome. After nuclease introduced DSBs, NHEJ repair will insert the tag sequence(s) into the target site, forming known primer landing sites. After cells have time to repair the DSBs and possibly further divide (such as after 72 hr), genomic DNA is isolated, fragmented (e.g., Covaris® shearing, enzyme-based shearing, Tn5, etc.), ligated a unique molecular index (UMI)-containing universal adapter sequence to the fragmented DNA, and the un-ligated material is removed. Next, the DNA fragments are amplified by targeting primers to the tag and universal adapter sequences (Round 1 PCR). Using universal primers, a sample index (PCR2) is added, the amplified material is concentration normalized, pooled with other samples, and the pooled material is sequenced on an IIlumina® (or similar) machine. The sequenced reads are aligned to a reference genome, and loci where large numbers of reads map may nominate on/off-target locations.

Alien sequences were designed by generating >1 M random 13-mer sequences with 40-90% GC content, max homopolymer length A:2, C:3, G:2, T:2, weighted homopolymer rate <20, self-folding Tₘ<50° C., and self-dimer Tₘ<50° C. From the list of sequences, sequences that aligned perfectly against human (GRCh38.p2; hg38) or mouse (GRCh38.p4; mm38) reference genomes or had troubling motif sequences (homopolymers, most G-G or C-C dinucleotide motifs) were removed, resulting in 479 sequences.

To design the 52-base pair tag sequences described herein, 49 13-mer oligo sequences were selected that contain ≤1 C or G dinucleotide, and 10,000 unique combinations of four 13-mer sequences were generated. The length of each concatenated sequence (e.g., pasting four 13-mer sequences in a row using software) is 52-nucleotides. Next, each 52-nucleotide tag sequence was aligned against the human (GRCh38.p2) and mouse (GRChm38.p4) genomes using an internally modified version of bwa, called bwa-psm. Implementation of bwa-psm returns all possible secondary matches up to a defined threshold. A set of tag sequences (SEQ ID NO:1-2) were designed that were intended to work as a group, that had no similarity to the human or mouse genomes (max seed size: 7, seed edit distance: 2, max edit distance: 21, max gap open: 2, max gap extension: 3, mismatch penalty: 1, gap open penalty: 1, gap extension penalty: 1).

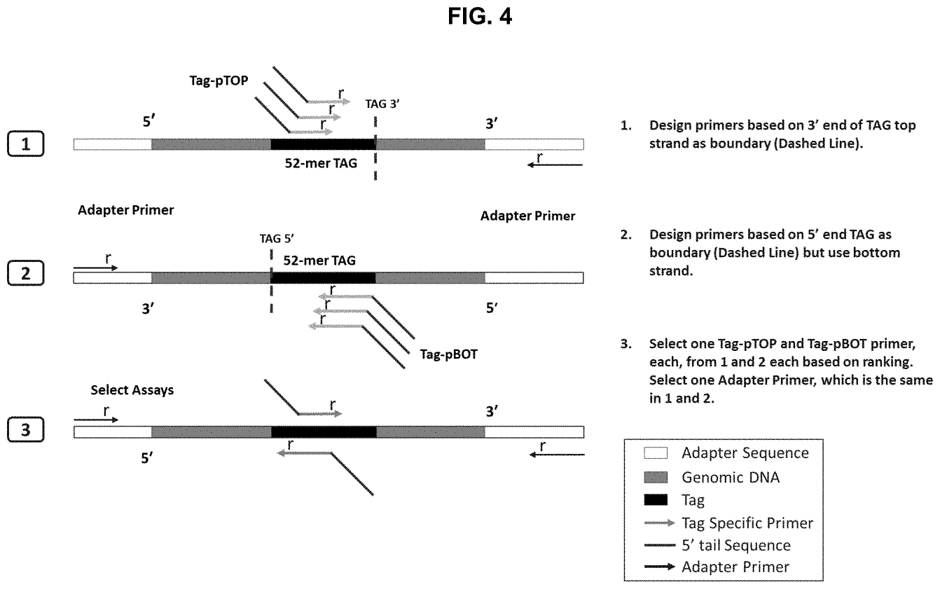

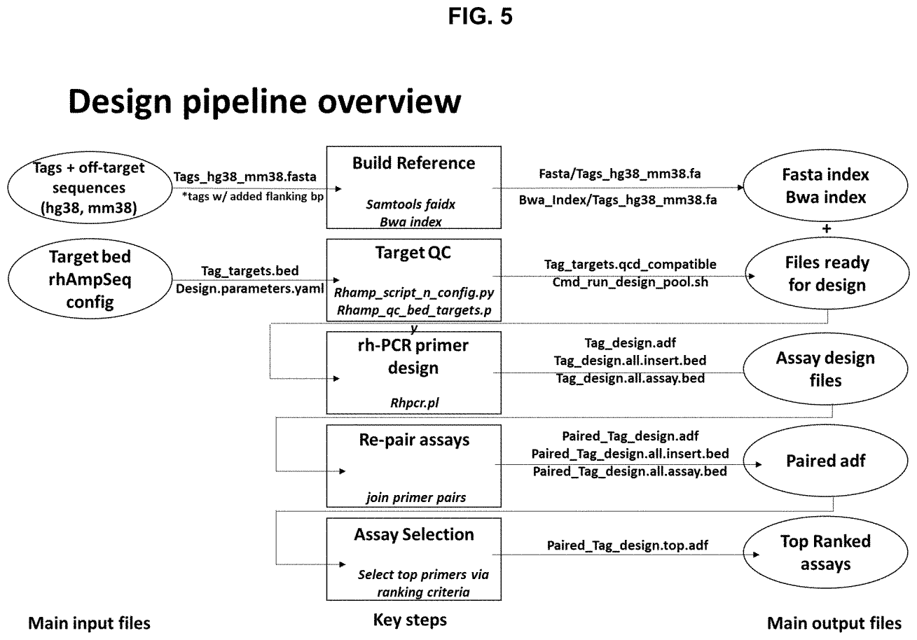

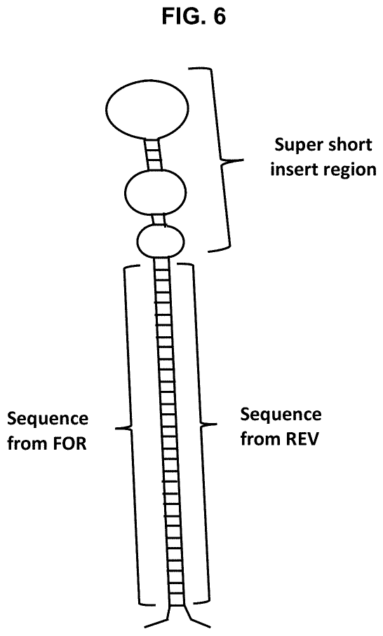

Overlapping rhAmpSeq V1 primers (SEQ ID NO: 3-4) were designed complementary to the top and bottom strands of the tag and 5′-end of the adapter sequence (SEQ ID NO: 6) (FIG. 4). The tag-specific primers (SEQ ID NO: 3-4) contain a 5′-universal tail sequence matching the SP1 and SP2 primer sequences (SEQ ID NO: 7-8), a locus specific segment, a ribonucleotide (rN) 6-nucleotides from the 3′-end, a 3′-end mismatch, and a 3′-end block (3′-C₃ spacer). The adapter-specific primer (SEQ ID NO: 5) targets the 5′-end of the 5′-P5 adapter sequence (SEQ ID NO: 6), and the adapter sequence contains unique molecular index (UMI) sequence (Table 2). The primers were designed to target the plus and minus strands of the annealed tag such that, if these primers unexpectedly form a dimer, the formed product will hairpin, removing the oligo from the available reaction templates (e.g., supression PCR). (FIG. 6A-B). Primer sequences targeting the tags were chosen based on a proprietary design algorithm designed and implemented by IDT (internal copy of the algorithm with a public-facing UI: www.idtdna.com/site/account?RetumURL=/site/order/designtool/index/RHAMPSEQ), which selects the most optimally performing primer pairs to amplify the intended template sequence. (FIG. 5). Primer sequences were assessed for non-specific binding to all other tag sequences and both human and mouse primary genome assemblies to verify they were unlikely to form off-target amplicons when combined with a universal adapter sequence and the presence of human or mouse genomic DNA.

The primers were desired to work in pairs where one tag-specific primer (top or bottom strand) pairs with the adapter-specific primer (SEQ ID NO:5). This results in the amplification of a molecule that contains a portion of the tag, gDNA, and the adapter sequence when amplified using supression PCR methods (FIG. 4).

TABLE 2
Sequences Used for First Proof of Concept

| | | | SEQ |
|---|---|---|---|
| | | Sequence | ID |
| Type | Name | (5′→3′) | NO |
| Tag | 9022179029169042579 | T*C*GTTCGTTC | SEQ |
| | 04625907201907281 | CGCTCTAACCGG | ID |
| | | CGAATCTACCGC | NO: |
| | | GCATATCTACGC | 1 |
| | | CGCA*A*T | |
| Tag | 9022179029169042579 | A*T*TGCGGCGT | SEQ |
| | 04625907201907281_r | AGATATGCGCGG | ID |
| | ev | TAGATTCGCCGG | NO: |
| | | TTAGAGCGGAAC | 2 |
| | | GAAC*G*A | |
| Tag | pFWD.ID_Target1: | acactctttccc | SEQ |
| Primers | 9022179029169042579 | tacacgacgctc | ID |
| | 04625907201907281.12 | ttccgatctTCT | NO: |
| | 7.150.1.SP1 | ACCGCGCATATC | 3 |
| | | TACrGCCGCT/ | |
| | | 3SpC3/ | |
| Tag | pFWD.ID_Target2: | acactctttccc | SEQ |
| Primers | 9022179029169042579 | tacacgacgctc | ID |
| | 04625907201907281.11 | ttccgatctATA | NO: |
| | 6.140.-1.SP1 | TGCGCGGTAGAT | 4 |
| | | TCGCrCGGTTT/ | |
| | | 3SpC3/ | |
| Adapter | Adapter Primer | gtgactggagtt | SEQ |
| Primer | | cagacgtgtgct | ID |
| | | cttccgatctAA | NO: |
| | | TGATACGGCGAC | 5 |
| | | CACCGAGATCTA | |
| | | CArCAAGGC/ | |
| | | 3SpC3/ | |
| P5 Adapter | Example Sequence | AATGATACGGCG | SEQ |
| | | ACCACCGAGATC | ID |
| | | TACACTAGATCG | NO: |
| | | CNNWNNWNNACA | 6 |
| | | CTCTTTCCCTAC | |
| | | ACGACGCTCTTC | |
| | | CGATC*T | |
| SP1 | Sequencing Primer 1 | acactctttccc | SEQ |
| | | tacacgacgctc | ID |
| | | ttccgatct | NO: |
| | | | 7 |
| SP2 | Sequencing Primer 2 | gtgactggagtt | SEQ |
| | | cagacgtgtgct | ID |
| | | cttccgatct | NO: |
| | | | 8 |

“*” indicates a phosphorothioate linkage; “rN” indicates a ribonucleotide, where N is the nucleotide preceeded by the “r”; “/3SpC3/” indicates a 3′-C₃ spacer.

One embodiment described herein is a method for identifying and identifying and nominating on- and off-target CRISPR editing sites with improved accuracy and sensitivity, the process comprising the steps of: (a) co-delivering a guide sequence RNA (sgRNA) or a two-part CRISPR RNA:trans-activating crRNA (crRNA:tracrRNA) duplex and one or more tag sequences to cells; (b) incubating the cells for a period of time; (c) isolating genomic DNA from the cells, fragmenting the genomic DNA, and ligating the fragmented genomic DNA to a unique molecular index containing a universal adapter sequence; (d) amplifying the ligated DNA fragments using primers targeting the tag and universal adapter sequences to produce a first set of amplified sequences; (e) amplifying the first set of amplified sequences using universal sequencing primers targeting the tails of Tag-pTOP or Tag-pBOT primers to produce a second set of amplified sequences; (f) sequencing the pooled sequences and obtaining sequencing data; and (g) identifying on-/off-target CRISPR editing loci. In one embodiment, the universal sequencing primers target SP1 or SP2 sequence (SEQ ID NO: 7, 8) tails on the Tag-pTOP or Tag-pBOT primers to produce a second set of amplified sequences. In another embodiment, the universal sequencing primers target predesigned non-homologous sequence (Table 6; SEQ ID NO: 269-273) tails on the Tag-pTOP or Tag-pBot to produce a second set of amplified sequences. In yet another embodiment, the universal primers target predesigned 13-mer tails on the Tag-pTOP or Tag-pBOT primers to produce a second set of amplified sequences. In one embodiment, step (g) comprises executing on a processor: (i) aligning the sequence data to a reference genome; (ii) identifying on-/off-target CRISPR editing loci; and (iii) outputting the alignment, analysis, and results data as tables or graphics. In another embodiment, the method further comprises a step following step (e) comprising: (e1) normalizing the second set of amplified sequences to produce concentration normalized libraries, pooling the normalized libraries with other samples to produce pooled libraries; and continuing with steps (f)-(i). In one aspect, step (d) uses a supression PCR method. In another aspect, the cells constitutively express a Cas enzyme, are co-delivered with a Cas expression vector, are co-delivered with a Cas protein, or are co-delivered with a Cas RNP complex. In another aspect, the cells constitutively express a Cas9 enzyme, are co-delivered with a Cas9 expression vector, are co-delivered with a Cas9 protein, or are co-delivered with a Cas9 RNP complex. In another aspect, the cells comprise human or mouse cells. In another aspect, the period of time is about 24 hours to about 96 hours. In another aspect, multiple tag sequences are co-delivered. In another aspect, the tag sequences comprise double-stranded deoxyribooligonucleotides (dsDNA) comprising 52-base pairs. In another aspect, the tag sequences comprise a 5′-terminal phosphate, and phosphorothioate linkages between the 1^{st} and 2^{nd}, 2^{nd} and 3_{rd}, 50^{th} and 51^{st}, and 51^{st} and 52^{nd} nucleotides. In another aspect, the tag sequences comprise a double stranded DNA comprising the top and bottom strand pairs of SEQ ID NO: 9-40 or 45-268.

Another embodiment described herein is on- and off-target CRISPR editing sites identified or nominated using the methods described herein.

Another embodiment described herein is a method for designing 52-base pair tag sequences, the method comprising, executing on a processor: (a) randomly generating 13-nucleotide sequences with 40-90% GC content, max homopolymer length A:2, C:3, G:2, T:2, weighted homopolymer rate <20, self-folding Tₘ<50° C., and self-dimer Tₘ<50° C.; (b) removing sequences that perfectly align to a particular genome or that are homopolymers or GG or CC dinucleotide motifs and obtaining a set of 13-mers; (c) selecting a subset of the 13-mer sequences that contain one or less CC or GG dinucleotide motifs; (d) concatenating four of the of 13-mer subset sequences to form random 52-mer sequences; (e) aligning the random 52-mer sequences to a genome; (f) removing the random 52-mer sequences that have similarity to the genome to produce a subset of 52-mer sequences; and (h) outputting the subset of 52-mer sequences and generating the complementary strands to produce double stranded 52-base pair tag sequences. In one aspect, the genome is human or mouse. In one aspect, the 52-base pair tag sequences are not complementary to the genome. In another aspect, the method further comprises designing primers for the 52-base pair tag sequences. In another aspect, the 52-base pair tag sequences comprise a 5′-terminal phosphate, and phosphorothioate linkages between the 1^{st} and 2^{nd}, 2^{nd} and 3_{rd}, 50^{th} and 51^{st}, and 51^{st} and 52^{nd} nucleotides of the 52-base pair tag sequences. In another aspect, the method further comprises synthesising oligonucleotides comprising the 52-base pair tag sequences, the complement of the 52-base pair tag sequences, or primers for the 52-base pair tag sequences.

Another embodiment described herein is one or more 52-base pair tag sequences designed using the methods described herein. In one aspect, the 52-base pair tag sequence comprises a double stranded DNA comprising the complementary top and bottom strand pairs of SEQ ID NO: 9-40 or 45-268.

Another embodiment described herein is a method for designing primers partially complementary to the 52-base pair tag sequences described herein and an adapter primer, the method comprising, executing on a processor: (a) designing tag primers that are partially complementary to the top and bottom strands of tag sequences; and (b) designing an adapter primer that is partially complementary to the top strand of the adapter sequence; wherein: the tag primers comprise a 5′-universal tail sequence complementary to an SP1 or SP2 sequence (SEQ ID NO: 7, 8), a locus specific segment, a ribonucleotide (rN) 6-nucleotides from the 3′-end, a 3′-end mismatch, and a 3′-end block (3′-C₃ spacer); and the adapter primer comprises a sequence complementary to the SP1 or SP2 sequence (SEQ ID NO: 7, 8). In one aspect, the primers partially complementary to top and bottom strands of the tag sequences comprise a sequence complementary to the SP1 sequence and the adapter primer comprises a sequence complementary to the SP2 sequence; or the primers partially complementary to top and bottom strands of the tag sequences comprise a sequence complementary to the SP2 sequence and the adapter primer comprises a sequence complementary to the SP1 sequence. In another aspect, amplification of a nucleic acid molecule with the primers that are complementary to the top and bottom strands of tag sequences and primers that are complementary to the top strand of the adapter sequence produces a PCR product that comprises a portion of the tag sequence, a sgDNA sequence, and the adapter sequence. In another aspect, the method further comprises synthesising oligonucleotides comprising the sequences of the forward and reverse tag primers and the adapter primer.

In another embodiment described herein, the 52-base pair tag sequences and primers partially complementary to the 52-base pair tag sequences are designed and selected using an algorithm predicting whether the primers are likely to be partially complementary and have a propensity to form primer-dimers.

Another embodiment described herein is one or more primers partially complementary to the 52-base pair tag sequences and one or more adapter primers designed using the methods described herein. In one aspect, the primers partially complementary to the 52-base pair tag sequence comprise the sequences of SEQ ID NO: 3, 4; and the adapter primer comprises the sequence of SEQ ID NO:5.

Another embodiment described herein is the use of one or more double-stranded 52-base pair tag sequences for identifying on- and off-target CRISPR editing sites.

It will be apparent to one of ordinary skill in the relevant art that suitable modifications and adaptations to the compositions, formulations, methods, processes, and applications described herein can be made without departing from the scope of any embodiments or aspects thereof. The compositions and methods provided are exemplary and are not intended to limit the scope of any of the specified embodiments. All the various embodiments, aspects, and options disclosed herein can be combined in any variations or iterations. The scope of the methods and processes described herein include all actual or potential combinations of embodiments, aspects, options, examples, and preferences herein described. The methods described herein may omit any component or step, substitute any component or step disclosed herein, or include any component or step disclosed elsewhere herein. It should also be understood that embodiments may include and otherwise be implemented by a combination of various hardware, software, and electronic components. For example, various microprocessors and application specific integrated circuits (“ASICs”) can be utilized, as can software of a variety of languages. Also, servers and various computing devices can be used and can include one or more processing units, one or more computer-readable mediums, one or more input/output interfaces, and various connections (e.g., a system bus) connecting the components. Should the meaning of any terms in any of the patents or publications incorporated by reference conflict with the meaning of the terms used in this disclosure, the meanings of the terms or phrases in this disclosure are controlling. Furthermore, the specification discloses and describes merely exemplary embodiments. All patents and publications cited herein are incorporated by reference herein for the specific teachings thereof.

Various embodiments and aspects of the inventions described herein are summarized by the following clauses:
Clause 1. A method for identifying and nominating on- and off-target CRISPR edited sites with improved accuracy and sensitivity, the process comprising the steps of:
   (a) co-delivering a guide sequence RNA (sgRNA) or a two-part CRISPR RNA:trans-activating crRNA (crRNA:tracrRNA) duplex, one or more tag sequences, and an RNA-guided endonuclease to cells;
   (b) incubating the cells for a period of time sufficient for double strand breaks to occur; (c) isolating genomic DNA from the cells, fragmenting the genomic DNA, and ligating the fragmented genomic DNA to a unique molecular index containing a universal adapter sequence;
   (d) amplifying the ligated DNA fragments using primers targeting the tag and universal adapter sequences to produce a first set of amplified sequences;
   (e) amplifying the first set of amplified sequences using universal sequencing primers targeting the tails of Tag-pTOP or Tag-pBOT primers to produce a second set of amplified sequences;
   (f) sequencing the pooled sequences and obtaining sequencing data; and
   (g) identifying on-/off-target CRISPR editing loci.
Clause 2. The method of clause 1, wherein the universal sequencing primers target SP1 or SP2 sequence (SEQ ID NO: 7, 8) tails on the Tag-pTOP or Tag-pBOT primers to produce a second set of amplified sequences.
Clause 3. The method of clause 1 or 2, wherein the universal sequencing primers target predesigned non-homologous sequence (SEQ ID NO: 269-273) tails on the Tag-pTOP or Tag-pBot primers to produce a second set of amplified sequences.
Clause 4. The method of any one of clauses 1-3, wherein the universal sequencing primers target predesigned 13-mer tails on the Tag-pTOP or Tag-pBot primers to produce a second set of amplified sequences.
Clause 5. The method of any one of clauses 1-4, wherein step (g) comprises executing on a processor:
Clause 6. aligning the sequence data to a reference genome;
   (a) (ii) identifying on-/off-target CRISPR editing loci; and
   (b) (iii) outputting the alignment, analysis, and results data as custom-formatted files, tables or graphics.
Clause 7. The method of any one of clauses 1-5, further comprising a step following step (e) comprising:
   (a) (e1) normalizing the second set of amplified sequences to produce concentration normalized libraries, pooling the normalized libraries with other samples to produce pooled libraries; and continuing with steps (f)-(i).
Clause 8. The method of any one of clauses 1-6, wherein step (d) uses a supression PCR method.
Clause 9. The method of any one of clauses 1-7, wherein the RNA-guided endonuclease comprises an endogenously-expressed Cas enzyme, a Cas expression vector, a Cas protein, or a Cas RNP complex.
Clause 10. The method of any one of clauses 1-8, wherein the RNA-guided endonuclease comprises an endogenously-expressed Cas9 enzyme, a Cas9 expression vector, a Cas9 protein, or a Cas9 RNP complex.
Clause 11. The method of any one of clauses 1-9, wherein the cells comprise human or mouse cells.
Clause 12. The method of any one of clauses 1-10, wherein the period of time is about 24 hours to about 96 hours.
Clause 13. The method of any one of clauses 1-11, wherein multiple tag sequences are co-delivered.
Clause 14. The method of any one of clauses 1-12, wherein the tag sequences comprise double-stranded deoxyribooligonucleotides (dsDNA) comprising 52-base pairs.
Clause 15. The method of any one of clauses 1-13, wherein the tag sequences comprise a 5′-terminal phosphate, and phosphorothioate linkages between the 1^{st} and 2^{nd}, 2^{nd} and 3^{rd}, 50^{th} and 51^{st}, and 51^{st} and 52^{nd} nucleotides.
Clause 16. The method of any one of clauses 1-14, wherein the tag sequences comprise a double stranded DNA comprising the complementary top and bottom strand pairs of SEQ ID NO: 1-2 or 7-268.
Clause 17. On- and off-target CRISPR editing sites identified or nominated using the method of any one of clauses 1-15.
Clause 18. A method for designing 52-base pair tag sequences, the method comprising, executing on a processor:
   (a) randomly generating 13-nucleotide sequences with 40-90% GC content, max homopolymer length A:2, C:3, G:2, T:2, weighted homopolymer rate <20, self-folding Tₘ<50° C., and self-dimer Tₘ<50° C.;
   (b) removing sequences that perfectly align to a particular genome or that are homopolymers or GG or CC dinucleotide motifs and obtaining a set of 13-mers;
   (c) selecting a subset of the 13-mer sequences that contain one or less CC or GG dinucleotide motifs;
   (d) concatenating four of the of 13-mer subset sequences to form random 52-mer sequences;
   (e) aligning the random 52-mer sequences to a genome;
   (f) removing the random 52-mer sequences that have similarity to the genome to produce a subset of 52-mer sequences; and
   (g) outputting the subset of 52-mer sequences and generating the complementary strands to produce double stranded 52-base pair tag sequences.
Clause 19. The method of clause 17, wherein the genome is human or mouse.
Clause 20. The method of clause 17 or 18, wherein the 52-base pair tag sequences are-non complementary to the genome.
Clause 21. The method of any one of clauses 17-19, further comprising designing primers for the 52-base pair tag sequences.
Clause 22. The method of any one of clauses 17-20, wherein the 52-base pair tag sequences comprise a 5′-terminal phosphate, and phosphorothioate linkages between the 1^{st} and 2^{nd}, 2^{nd} and 3^{rd}, 50^{th} and 51^{st}, and 51^{st} and 52^{nd} nucleotides of the 52-base pair tag sequences.
Clause 23. The method of any one of clauses 17-21, further comprising synthesizing oligonucleotides comprising the 52-base pair tag sequences, the complement of the 52-base pair tag sequences, or primers for the 52-base pair tag sequences.
Clause 24. One or more 52-base pair tag sequences designed using the methods of clauses 17-22.
Clause 25. The 52-base pair tag sequences of clause 23, wherein the 52-base pair tag sequence comprises a double stranded DNA comprising the top and bottom strand pairs of SEQ ID NO: 1-2 or 7-268.
Clause 26. A method for designing primers partially complementary to the 52-base pair tag sequences of clause 23 and an adapter primer, the method comprising, executing on a processor:
   (a) designing tag primers that are partially complementary to the top and bottom strands of tag sequences; and
   (b) designing an adapter primer that is partially complementary to the top strand of the adapter sequence;
   (c) wherein:
   (d) the tag primers comprise a 5′-universal tail sequence; and
   (e) the adapter primer comprises a sequence complementary to the tails of Tag-pTOP or Tag-pBOT primers.
Clause 27. The method of clause 25, wherein the 5′-universal tail sequence is complementary to an SP1 or SP2 sequence (SEQ ID NO: 7, 8), a locus specific segment, a ribonucleotide (rN) 6-nucleotides from the 3′-end, a 3′-end mismatch, a 3′-end block (3′-C₃ spacer), a predesigned non-homologous sequence (SEQ ID NO: 269-273), or a predesigned 13-mer sequence.
Clause 28. The method of clause 25 or 26, wherein the primers partially complementary to top and bottom strands of the tag sequences comprise a tail sequence complementary to the SP1 sequence (SEQ ID NO: 7) and the adapter primer comprises a sequence complementary to the SP2 sequence (SEQ ID NO: 8) tail on the Tag-pTOP or Tag-pBOT primers; or the primers partially complementary to top and bottom strands of the tag sequences comprise a tail sequence complementary to the SP2 sequence (SEQ ID NO: 8) and the adapter primer comprises a sequence complementary to the SP1 sequence (SEQ ID NO: 7) tail on the Tag-pTOP or Tag-pBOT primers.
Clause 29. The method of any one of clauses 25-27, wherein the amplification of a nucleic acid molecule with the primers that are complementary to the top and bottom strands of tag sequences and primers that are complementary to the top strand of the adapter sequence produces a PCR product that comprises a portion of the tag sequence, a sgDNA sequence, and the adapter sequence.
Clause 30. The method of any one of clauses 25-28, further comprising synthesizing oligonucleotides comprising the sequences of the forward and reverse tag primers and the adapter primer.
Clause 31. The method of any one of clauses 17-21 and 25-29, wherein the 52-base pair tag sequences and primers partially complementary to the 52-base pair tag sequences are designed and selected using an algorithm predicting whether the primers are likely to be partially complementary and have a propensity to form primer-dimers.
Clause 32. One or more primers partially complementary to the 52-base pair tag sequences and one or more adapter primers designed using the method of clauses 22-25.
Clause 33. The primers of clause 32, wherein the primers comprise the sequences of SEQ ID NO: 3, 4; and the adapter primer, wherein the adapter primer comprises the sequence of SEQ ID NO: 5.
Clause 34. Use of one or more double-stranded 52-base pair tag sequences for identifying on- and off-target CRISPR editing sites.

### REFERENCES

1. Wenert et al., “Unbiased detection of CRISPR off-targets in vivo using DISCOVER-seq,” Science 364(6437): 286-289 (2019).
2. Nobles et al., “IGUIDE: An improved pipeline for analyzing CRISPR cleavage specificity,” Genome Biol. 20(14): 4-9 (2019).
3. Tsai et al., “GUIDE-seq enables genome-wide profiling of off-target cleavage by CRISPR-Cas nucleases,” Nature Biotechnol. 33(2): 187-197 (2015).
4. Yan et al., “BLISS is a versatile and quantitative method for genome-wide profiling of DNA double-strand breaks,” Nature Commun. 8: 15058 (2017).
5. Tsai et al., “CIRCLE-seq: a highly sensitive in vitro screen for genome-wide CRISPR-Cas9 nuclease off-targets,” Nature Methods 14(6): 607-614 (2017).
6. Cameron et al., “Mapping the genomic landscape of CRISPR-Cas9 cleavage,” Nature Methods 14(6): 600-606 (2017).
7 Char and Moosburner, “Unraveling CRISPR-Cas9 genome engineering parameters via a library-on-library approach,” Nature Methods 12(9): 823-826 (2015).
8. Rand et al., “Headloop suppression PCR and its application to selective amplification of methylated DNA sequences,” Nucleic Acids Res. 33(14):e127 (2005).

### EXAMPLES

### Example 1

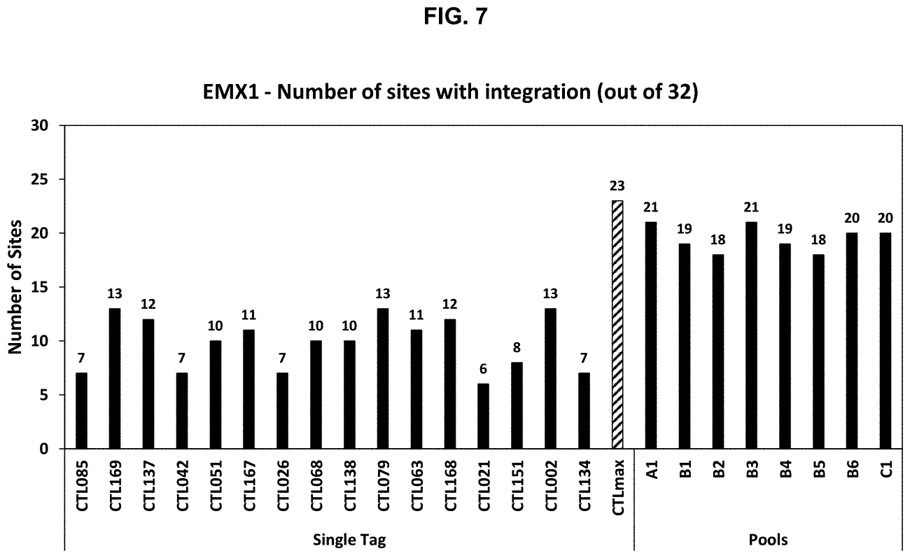

This experiment demonstrates the increased efficiency in tag integration when using double-stranded DNA tags with a length of 52-base pairs and varying genetic sequence. The sequences used are shown in Tables 3-5. Double-stranded tags were generated by hybridization of a top strand and a complementary bottom strand (Tables 3-4; SEQ ID NO: 9-40 or 45-268). Sixteen different tag designs were introduced separately into HEK293 cells constitutively expressing Cas9 together with a guideRNA which targets the EMX1 locus. Alternatively, either pools of 16 tags or one pool of 112 tags were introduced into HEK293 cells constitutively expressing Cas9 together with a guideRNA which targets the EMX1 locus. GuideRNAs were electroporated at a concentration of 10 μM, whereas the single Tag or pooled Tags were delivered at a final concentration of 0.5 μM. Tag integration levels were determined by targeted amplification using rhAmpSeq primers (SEQ ID NO: 3-4), enriching for known on- and off-target sites of the EMX1 guideRNA. The rhAmpSeq pool for EMX1 consists of 32 sites, which represent empirically determined ON and OFF target loci. Amplified products were sequenced on an Illumina® MiSeq, and tag integration levels were determined using custom software. This example shows that tag integration efficiency varies among single tag constructs individually with a range between 6 (CTL021) and 13 (CTL169, CTL079, CTL002) sites out of a maximum of 32 sites, and is therefore sequence dependent (Single Tags, FIG. 7). By taking the mathematical union of the single tag results, a hypothetical number of 23 sites was calculated (CTLmax, FIG. 7). The hypothesis that combining a pool of tags would increase the likelihood of tag integration was tested and was demonstrated (Pooled Tags, Table, FIG. 7). Pool A1 consists of the tags represented in the Single Tags (see Table 5) and demonstrated that 21 tag integration events were detected out of a maximum of 32 sites, which is higher than achieved with any of the single tags. Similarly, Pool B3 demonstrated integration of a tag at 21 sites out of a maximum of 32 sites. Again, variability between pools was shown (Pooled Tags, FIG. 7), indicating optimization of tag designs can potentially maximize tag integration.

TABLE 3
Sequences Used for Second Proof of
Concept

| | | SEQ |
|---|---|---|
| | | ID |
| Name | Sequence (5′→3′) | NO |
| CTL085_ | /5Phos/A*C*GAGCGGTAGTCACCTA | SEQ |
| TOP_tag | GTCGTCGTACCAATTCGACGCACACTA | ID |
| | CTCGC*G*C | NO: |
| | | 9 |
| CTL085_ | /5Phos/G*C*GCGAGTAGTGTGCGTC | SEQ |
| BOT_tag | GAATTGGTACGACGACTAGGTGACTAC | ID |
| | CGCTC*G*T | NO: |
| | | 10 |
| CTL169_ | /5Phos/T*A*GCGCGAGTAGTCGGAC | SEQ |
| TOP_tag | GAGCGGTTACCAATACGCCGCACCTTA | ID |
| | ATCCG*C*G | NO: |
| | | 11 |
| CTL169_ | /5Phos/C*G*CGGATTAAGGTGCGGC | SEQ |
| BOT_tag | GTATTGGTAACCGCTCGTCCGACTACT | ID |
| | CGCGC*T*A | NO: |
| | | 12 |
| CTL137_ | /5Phos/T*C*GCGACAGTAGTCGTTC | SEQ |
| TOP_tag | GGCTAGGTACCTATTACCGCGTAGTTA | ID |
| | GCGGC*G*T | NO: |
| | | 13 |
| CTL137_ | /5Phos/A*C*GCCGCTAACTACGCGG | SEQ |
| BOT_tag | TAATAGGTACCTAGCCGAACGACTACT | ID |
| | GTCGC*G*A | NO: |
| | | 14 |
| CTL042_ | /5Phos/C*G*CGCTACTAGGTGCGTC | SEQ |
| TOP_tag | GAATTGGTACCGATCCGCAATACACTA | ID |
| | CTCGC*G*C | NO: |
| | | 15 |
| CTL042_ | /5Phos/G*C*GCGAGTAGTGTATTGC | SEQ |
| BOT_tag | GGATCGGTACCAATTCGACGCACCTAG | ID |
| | TAGCG*C*G | NO: |
| | | 16 |
| CTL051_ | /5Phos/G*G*TAACGAGCGGTGCGTC | SEQ |
| TOP_tag | GAATTGGTAACCGCTCGTCCGACCTTA | ID |
| | ATCGC*G*C | NO: |
| | | 17 |
| CTL051_ | /5Phos/G*C*GCGATTAAGGTCGGAC | SEQ |
| BOT_tag | GAGCGGTTACCAATTCGACGCACCGCT | ID |
| | CGTTA*C*C | NO: |
| | | 18 |
| CTL167_ | /5Phos/T*T*CGGCGCTAGGTGCGGC | SEQ |
| TOP_tag | GTATTGGTAACCGCTCGTCCGTTCGGC | ID |
| | GCTAG*G*T | NO: |
| | | 19 |
| CTL167_ | /5Phos/A*C*CTAGCGCCGAACGGAC | SEQ |
| BOT_tag | GAGCGGTTACCAATACGCCGCACCTAG | ID |
| | CGCCG*A*A | NO: |
| | | 20 |
| CTL026_ | /5Phos/T*A*CGCGACTAGGTGCGCG | SEQ |
| TOP_tag | ATTAAGGTACCTATTACCGCGCGACTA | ID |
| | TGTGC*G*C | NO: |
| | | 21 |
| CTL026_ | /5Phos/G*C*GCACATAGTCGCGCGG | SEQ |
| BOT_tag | TAATAGGTACCTTAATCGCGCACCTAG | ID |
| | TCGCG*T*A | NO: |
| | | 22 |
| CTL068_ | /5Phos/G*T*CGCGCAGTGTAGCGCG | SEQ |
| TOP_tag | ATTAAGGTACCTATTACCGCGTCGCGA | ID |
| | CAGTA*G*T | NO: |
| | | 23 |
| CTL068_ | /5Phos/A*C*TACTGTCGCGACGCGG | SEQ |
| BOT_tag | TAATAGGTACCTTAATCGCGCTACACT | ID |
| | GCGCG*A*C | NO: |
| | | 24 |
| CTL138_ | /5Phos/A*A*CCGTCGATCCGCGCGT | SEQ |
| TOP_tag | AGTATGGTACCGATCCGCAATACTAGC | ID |
| | GCGAC*A*A | NO: |
| | | 25 |
| CTL138_ | /5Phos/T*T*GTCGCGCTAGTATTGC | SEQ |
| BOT_tag | GGATCGGTACCATACTACGCGCGGATC | ID |
| | GACGG*T*T | NO: |
| | | 26 |
| CTL079_ | /5Phos/T*C*GCTCGATTGGTTACGC | SEQ |
| TOP_tag | GCACTACTTATGCGCTCGACTCGTTCG | ID |
| | GCTAG*G*T | NO: |
| | | 27 |
| CTL079_ | /5Phos/A*C*CTAGCCGAACGAGTCG | SEQ |
| BOT_tag | AGCGCATAAGTAGTGCGCGTAACCAAT | ID |
| | CGAGC*G*A | NO: |
| | | 28 |
| CTL063_ | /5Phos/A*C*TGCGAGCGTACTTGTC | SEQ |
| TOP_tag | GCGCTAGTACCAATTCGACGCAACCGC | ID |
| | TCGTC*C*G | NO: |
| | | 29 |
| CTL063_ | /5Phos/C*G*GACGAGCGGTTGCGTC | SEQ |
| BOT_tag | GAATTGGTACTAGCGCGACAAGTACGC | ID |
| | TCGCA*G*T | NO: |
| | | 30 |
| CTL168_ | /5Phos/C*G*CATTAGTCGGTGCGGC | SEQ |
| TOP_tag | GTATTGGTAACCGCTCGTCCGACGCGC | ID |
| | TACCT*A*T | NO: |
| | | 31 |
| CTL168_ | /5Phos/A*T*AGGTAGCGCGTCGGAC | SEQ |
| BOT_tag | GAGCGGTTACCAATACGCCGCACCGAC | ID |
| | TAATG*C*G | NO: |
| | | 32 |
| CTL021_ | /5Phos/A*T*TGCGGATCGGTGCGTC | SEQ |
| TOP_tag | GAATTGGTAACCGCTCGTCCGTACGCG | ID |
| | CACTA*C*T | NO: |
| | | 33 |
| CTL021_ | /5Phos/A*G*TAGTGCGCGTACGGAC | SEQ |
| BOT_tag | GAAGCGGTTACCAATTCGCGCACCGAT | ID |
| | CCGCA*A*T | NO: |
| | | 34 |
| CTL151_ | /5Phos/T*C*GGCGAGTAGTTGCGCG | SEQ |
| TOP_tag | GTTATGGTACCATAACCGCGCAGTAGT | ID |
| | ACGCG*G*T | NO: |
| | | 35 |
| CTL151_ | /5Phos/A*C*CGCGTACTACTGCGCG | SEQ |
| BOT_tag | GTTATGGTACCATAACCGCGCAACTAC | ID |
| | TCGCC*G*A | NO: |
| | | 36 |
| CTL002_ | /5Phos/A*C*TAGCGATCGGTACCTA | SEQ |
| TOP_tag | GCGCCGAAACCTATTACCGCGACCTAG | ID |
| | CGTTG*C*G | NO: |
| | | 37 |
| CTL002_ | /5Phos/C*G*CAACGCTAGGTCGCGG | SEQ |
| BOT_tag | TAATAGGTTTCGGCGCTAGGTACCGAT | ID |
| | CGCTA*G*T | NO: |
| | | 38 |
| CTL134_ | /5Phos/T*A*GCGCGTCAAGAGCGCG | SEQ |
| TOP_tag | GTTATGGTTTCGGCGCTAGGTTAACAG | ID |
| | CGCGT*C*G | NO: |
| | | 39 |
| CTL134_ | /5Phos/C*G*ACGCGCTGTTAACCTA | SEQ |
| BOT_tag | GCGCCGAAACCATAACCGCGCTCTTGA | ID |
| | CGCGC*T*A | NO: |
| | | 40 |
| GuideSeq_ | /5Phos/G*T*TTAATTGAGTTGTCAT | SEQ |
| TOP_tag | ATGTTAATAACGGT*A*T | ID |
| | | NO: |
| | | 41 |
| GuideSeq_ | /5Phos/A*T*ACCGTTATTAACATAT | SEQ |
| BOT_tag | GACAACTCAATTAA*A*C | ID |
| | | NO: |
| | | 42 |
| EMX1 | GAGTCCGAGCAGAAGAAGAA | SEQ |
| protospacer | | ID |
| | | NO: |
| | | 43 |
| AR | GTTGGAGCATCTGAGTCCAG | SEQ |
| protospacer | | ID |
| | | NO: |
| | | 44 |

“/5Phos/” indicates a 5′-phosphate moiety; “*” indicates a phosphorothioate linkage.

### Example 2

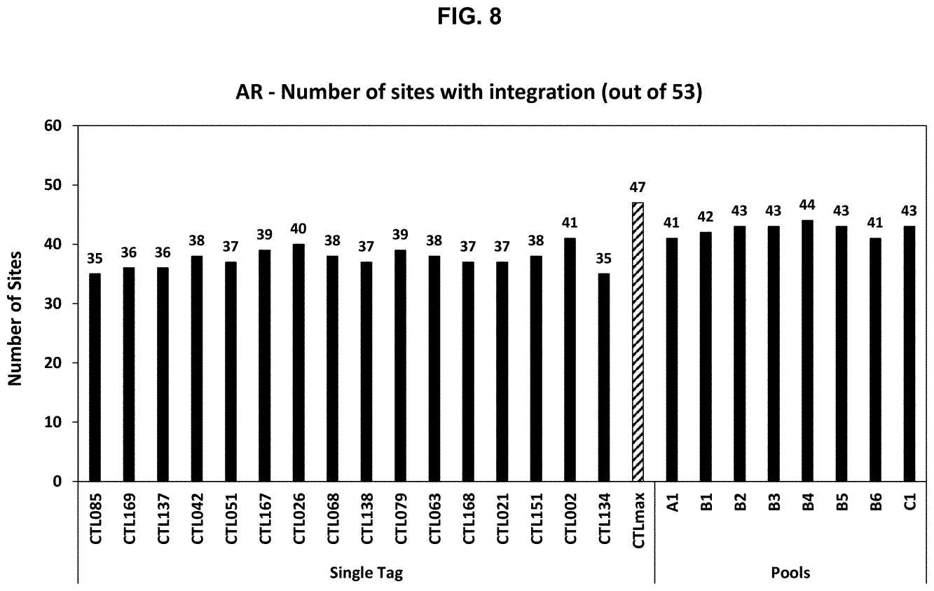

This experiment demonstrates the increased efficiency in tag integration when using double-stranded DNA tags with a length of 52-base pairs and varying genetic sequence. The sequences used are shown in Tables 3-5. Double-stranded tags were generated by hybridization of a top strand and a complementary bottom strand (SEQ ID NO: 9-40 or 45-268). Sixteen different tag designs were introduced separately into HEK293 cells constitutively expressing Cas9 together with a guideRNA which targets the AR locus. Alternatively, either pools of 16 tags or one pool of 112 tags were introduced into HEK293 cells constitutively expressing Cas9 together with a guideRNA which targets the AR locus. GuideRNAs were electroporated at a concentration of 10 μM, whereas the single Tag or pooled Tags were delivered at a final concentration of 0.5 μM. Tag integration levels were determined by targeted amplification using rhAmpSeq primers (SEQ ID NO: 3-4), enriching for known on- and off-target sites of the AR guideRNA. The rhAmpSeq pool for AR consists of 53 sites which represent empirically determined ON and OFF target loci. Amplified products were sequenced on an Illumina® MiSeq, and tag integration levels were determined using custom software. This example shows that tag integration efficiency varies among single tag constructs individually with a range between 35 (CTL085, CTL134) and 41 sites (CTL002) out of a maximum of 53 sites, and is therefore sequence dependent (Single Tags, Table 5, FIG. 8).

By taking the mathematical union of the single tag results, a hypothetical number of 47 sites was calculated (CTLmax, FIG. 8). The hypothesis that combining a pool of tags would increase the likelihood of tag integration was tested and was demonstrated (Pooled Tags, Table 5, FIG. 8). Pool B4 (see Table 5) demonstrated that 44 tag integration events were detected out of a maximum of 53 sites, which is higher than achieved with any of the single tags. Again, variability between pools was shown (Pooled Tags, Table 5, FIG. 8), indicating optimization of tag designs can potentially maximize tag integration.

TABLE 4
Tag Sequences

| Name | Sequence (5′→3′) | SEQ ID NO |
|---|---|---|
| CTL085_TOP_tag | /5Phos/A*C*GAGCGGTAGTCACCTAGTCGTCGTACCAATTCGA | SEQ ID NO: 45 |
| | CGCACACTACTCGC*G*C | |
| CTL169_TOP_tag | /5Phos/T*A*GCGCGAGTAGTCGGACGAGCGGTTACCAATACGC | SEQ ID NO: 46 |
| | CGCACCTTAATCCG*C*G | |
| CTL137_TOP_tag | /5Phos/T*C*GCGACAGTAGTCGTTCGGCTAGGTACCTATTACC | SEQ ID NO: 47 |
| | GCGTAGTTAGCGGC*G*T | |
| CTL042_TOP_tag | /5Phos/C*G*CGCTACTAGGTGCGTCGAATTGGTACCGATCCGC | SEQ ID NO: 48 |
| | AATACACTACTCGC*G*C | |
| CTL051_TOP_tag | /5Phos/G*G*TAACGAGCGGTGCGTCGAATTGGTAACCGCTCGT | SEQ ID NO: 49 |
| | CCGACCTTAATCGC*G*C | |
| CTL167_TOP_tag | /5Phos/T*T*CGGCGCTAGGTGCGGCGTATTGGTAACCGCTCGT | SEQ ID NO: 50 |
| | CCGTTCGGCGCTAG*G*T | |
| CTL026_TOP_tag | /5Phos/T*A*CGCGACTAGGTGCGCGATTAAGGTACCTATTACC | SEQ ID NO: 51 |
| | GCGCGACTATGTGC*G*C | |
| CTL068_TOP_tag | /5Phos/G*T*CGCGCAGTGTAGCGCGATTAAGGTACCTATTACC | SEQ ID NO: 52 |
| | GCGTCGCGACAGTA*G*T | |
| CTL138_TOP_tag | /5Phos/A*A*CCGTCGATCCGCGCGTAGTATGGTACCGATCCGC | SEQ ID NO: 53 |
| | AATACTAGCGCGAC*A*A | |
| CTL079_TOP_tag | /5Phos/T*C*GCTCGATTGGTTACGCGCACTACTTATGCGCTCG | SEQ ID NO: 54 |
| | ACTCGTTCGGCTAG*G*T | |
| CTL063_TOP_tag | /5Phos/A*C*TGCGAGCGTACTTGTCGCGCTAGTACCAATTCGA | SEQ ID NO: 55 |
| | CGCAACCGCTCGTC*C*G | |
| CTL168_TOP_tag | /5Phos/C*G*CATTAGTCGGTGCGGCGTATTGGTAACCGCTCGT | SEQ ID NO: 56 |
| | CCGACGCGCTACCT*A*T | |
| CTL021_TOP_tag | /5Phos/A*T*TGCGGATCGGTGCGTCGAATTGGTAACCGCTCGT | SEQ ID NO: 57 |
| | CCGTACGCGCACTA*C*T | |
| CTL151_TOP_tag | /5Phos/T*C*GGCGAGTAGTTGCGCGGTTATGGTACCATAACCG | SEQ ID NO: 58 |
| | CGCAGTAGTACGCG*G*T | |
| CTL002_TOP_tag | /5Phos/A*C*TAGCGATCGGTACCTAGCGCCGAAACCTATTACC | SEQ ID NO: 59 |
| | GCGACCTAGCGTTG*C*G | |
| CTL134_TOP_tag | /5Phos/T*A*GCGCGTCAAGAGCGCGGTTATGGTTTCGGCGCTA | SEQ ID NO: 60 |
| | GGTTAACAGCGCGT*C*G | |
| CTL085_BOT_tag | /5Phos/G*C*GCGAGTAGTGTGCGTCGAATTGGTACGACGACTA | SEQ ID NO: 61 |
| | GGTGACTACCGCTC*G*T | |
| CTL169_BOT_tag | /5Phos/C*G*CGGATTAAGGTGCGGCGTATTGGTAACCGCTCGT | SEQ ID NO: 62 |
| | CCGACTACTCGCGC*T*A | |
| CTL137_BOT_tag | /5Phos/A*C*GCCGCTAACTACGCGGTAATAGGTACCTAGCCGA | SEQ ID NO: 63 |
| | ACGACTACTGTCGC*G*A | |
| CTL042_BOT_tag | /5Phos/G*C*GCGAGTAGTGTATTGCGGATCGGTACCAATTCGA | SEQ ID NO: 64 |
| | CGCACCTAGTAGCG*C*G | |
| CTL051_BOT_tag | /5Phos/G*C*GCGATTAAGGTCGGACGAGCGGTTACCAATTCGA | SEQ ID NO: 65 |
| | CGCACCGCTCGTTA*C*C | |
| CTL167_BOT_tag | /5Phos/A*C*CTAGCGCCGAACGGACGAGCGGTTACCAATACGC | SEQ ID NO: 66 |
| | CGCACCTAGCGCCG*A*A | |
| CTL026_BOT_tag | /5Phos/G*C*GCACATAGTCGCGCGGTAATAGGTACCTTAATCG | SEQ ID NO: 67 |
| | CGCACCTAGTCGCG*T*A | |
| CTL068_BOT_tag | /5Phos/A*C*TACTGTCGCGACGCGGTAATAGGTACCTTAATCG | SEQ ID NO: 68 |
| | CGCTACACTGCGCG*A*C | |
| CTL138_BOT_tag | /5Phos/T*T*GTCGCGCTAGTATTGCGGATCGGTACCATACTAC | SEQ ID NO: 69 |
| | GCGCGGATCGACGG*T*T | |
| CTL079_BOT_tag | /5Phos/A*C*CTAGCCGAACGAGTCGAGCGCATAAGTAGTGCGC | SEQ ID NO: 70 |
| | GTAACCAATCGAGC*G*A | |
| CTL063_BOT_tag | /5Phos/C*G*GACGAGCGGTTGCGTCGAATTGGTACTAGCGCGA | SEQ ID NO: 71 |
| | CAAGTACGCTCGCA*G*T | |
| CTL168_BOT_tag | /5Phos/A*T*AGGTAGCGCGTCGGACGAGCGGTTACCAATACGC | SEQ ID NO: 72 |
| | CGCACCGACTAATG*C*G | |
| CTL021_BOT_tag | /5Phos/A*G*TAGTGCGCGTACGGACGAGCGGTTACCAATTCGA | SEQ ID NO: 73 |
| | CGCACCGATCCGCA*A*T | |
| CTL151_BOT_tag | /5Phos/A*C*CGCGTACTACTGCGCGGTTATGGTACCATAACCG | SEQ ID NO: 74 |
| | CGCAACTACTCGCC*G*A | |
| CTL002_BOT_tag | /5Phos/C*G*CAACGCTAGGTCGCGGTAATAGGTTTCGGCGCTA | SEQ ID NO: 75 |
| | GGTACCGATCGCTA*G*T | |
| CTL134_BOT_tag | /5Phos/C*G*ACGCGCTGTTAACCTAGCGCCGAAACCATAACCG | SEQ ID NO: 76 |
| | CGCTCTTGACGCGC*T*A | |
| CTL161_TOP_tag | /5Phos/T*A*CACTGCGCGACACTGCGAGCGTACACCTTAATCG | SEQ ID NO: 77 |
| | CGCTAGTTAGCGGC*G*T | |
| CTL164_TOP_tag | /5Phos/A*A*CCGTCGAGTGCACCGCGTACTACTAATGTCGAAC | SEQ ID NO: 78 |
| | CGCTACGCGCACTA*C*T | |
| CTL030_TOP_tag | /5Phos/C*G*CGGACTAAGGTGCGCGAGTAGTGTTACGCGCACT | SEQ ID NO: 79 |
| | ACTAATCTAGCCGC*G*A | |
| CTL088_TOP_tag | /5Phos/A*C*TAGTGCGACGAACTACTCGCGCTAACCAATTCGA | SEQ ID NO: 80 |
| | CGCACCGATCGCTA*G*T | |
| CTL148_TOP_tag | /5Phos/A*A*TGTCGAACCGCGCGCGAGTAGTGTACCATAACCG | SEQ ID NO: 81 |
| | CGCACCTTAGTCCG*C*G | |
| CTL152_TOP_tag | /5Phos/G*C*GTCGAATTGGTACCGCCGACTTATACCAATACGC | SEQ ID NO: 82 |
| | CGCATAGGTAGCGC*G*T | |
| CTL007_TOP_tag | /5Phos/A*C*CTAGTAGCGCGGCGTCGAATTGGTACTAGCGCGA | SEQ ID NO: 83 |
| | CAACGCGTAGTATG*G*T | |
| CTL141_TOP_tag | /5Phos/A*C*CGCTCGTTACCGCGCGATTAAGGTACGCCGCTAA | SEQ ID NO: 84 |
| | CTACGGTACGGTCG*G*T | |
| CTL064_TOP_tag | /5Phos/A*C*CGCCGACTTATCGTTCGGCTAGGTACCAATTCGA | SEQ ID NO: 85 |
| | CGCACTGCGAGCGT*A*C | |
| CTL158_TOP_tag | /5Phos/A*C*CTTAATCCGCGACTGCGAGCGTACACCTATTACC | SEQ ID NO: 86 |
| | GCGCGACGCGCTGT*T*A | |
| CTL066_TOP_tag | /5Phos/A*C*GACGACTAGGTACCGCTCGTTACCTCTTGACGCG | SEQ ID NO: 87 |
| | CTAACCAATTCGAC*G*C | |
| CTL144_TOP_tag | /5Phos/A*C*CATACTACGCGGCGGTTCGACATTACCATAACCG | SEQ ID NO: 88 |
| | CGCTAGTGCGAGCG*T*A | |
| CTL107_TOP_tag | /5Phos/C*T*TGTACGGCGGTGCGGCGTATTGGTACCAATACGC | SEQ ID NO: 89 |
| | CGCTCGTCGCACTA*G*T | |
| CTL149_TOP_tag | /5Phos/G*T*ACGCTCGCAGTACCGCCGACTTATACCTTAATCG | SEQ ID NO: 90 |
| | CGCACTAGCGCGAC*A*A | |
| CTL008_TOP_tag | /5Phos/A*C*GACGACTAGGTTATGGTACGGCGTTAGCGCGAGT | SEQ ID NO: 91 |
| | AGTACCTTAGTCCG*C*G | |
| CTL099_TOP_tag | /5Phos/A*C*GAGCGGTAGTCATAGGTAGCGCGTTCTTGACGCG | SEQ ID NO: 92 |
| | CTAACCGATCGCTA*G*T | |
| CTL089_TOP_tag | /5Phos/A*C*CGATCCGCAATGCGTCGAATTGGTACCATAACCG | SEQ ID NO: 93 |
| | CGCACCGCCGTACA*A*G | |
| CTL081_TOP_tag | /5Phos/A*C*TAGTGCGACGAACTACTGTCGCGAACCTATTACC | SEQ ID NO: 94 |
| | GCGACCAATCGAGC*G*A | |
| CTL075_TOP_tag | /5Phos/A*C*CGCCGTACAAGTCGCGACAGTAGTAACCGCTCGT | SEQ ID NO: 95 |
| | CCGTTCGGCGCTAG*G*T | |
| CTL160_TOP_tag | /5Phos/T*C*GTCGCACTAGTCGCATTAGTCGGTAGTAGTACGC | SEQ ID NO: 96 |
| | GGTATAGGTAGCGC*G*T | |
| CTL133_TOP_tag | /5Phos/A*C*CAATTCGACGCTAGTTAGCGGCGTACACTACTCG | SEQ ID NO: 97 |
| | CGCGCACTCGACGG*T*T | |
| CTL076_TOP_tag | /5Phos/C*G*CGGTAATAGGTCGCGGTAATAGGTACGAGCGGTA | SEQ ID NO: 98 |
| | GTCACACTACTCGC*G*C | |
| CTL024_TOP_tag | /5Phos/T*C*GGCGAGTAGTTTAGTGCGAGCGTAAGTAGTGCGC | SEQ ID NO: 99 |
| | GTAACCAATCGAGC*G*A | |
| CTL045_TOP_tag | /5Phos/G*T*CGCGCAGTGTAGCGCGGTTATGGTACCATAACCG | SEQ ID NO: 100 |
| | CGCACTAGTGCGAC*G*A | |
| CTL009_TOP_tag | /5Phos/T*A*TGCGCTCGACTGCGCGATTAAGGTAATGTCGAAC | SEQ ID NO: 101 |
| | CGCAGTAGTACGCG*G*T | |
| CTL055_TOP_tag | /5Phos/A*C*TAGCGCGACAACGACTATGTGCGCACCAATTCGA | SEQ ID NO: 102 |
| | CGCTACGCGCACTA*C*T | |
| CTL101_TOP_tag | /5Phos/A*A*CTACTCGCCGACTTGTACGGCGGTACCAATTCGA | SEQ ID NO: 103 |
| | CGCAACTAATCCGC*G*C | |
| CTL135_TOP_tag | /5Phos/C*G*CGGATTAAGGTCTTGTACGGCGGTACCTAGCCGA | SEQ ID NO: 104 |
| | ACGTACGCGCACTA*C*T | |
| CTL155_TOP_tag | /5Phos/T*A*GCGCGTCAAGACTTGTACGGCGGTACCGATCCGC | SEQ ID NO: 105 |
| | AATGCACTCGACGG*T*T | |
| CTL122_TOP_tag | /5Phos/C*G*CATTAGTCGGTGCGGCGTATTGGTACGACGACTA | SEQ ID NO: 106 |
| | GGTACCAATACGCC*G*C | |
| CTL080_TOP_tag | /5Phos/A*C*CTAGTAGCGCGGCGCGGTTATGGTACCGACTAAT | SEQ ID NO: 107 |
| | GCGACTAGCGATCG*G*T | |
| CTL126_TOP_tag | /5Phos/A*C*TACTCGCGCTAACCTAGTCGTCGTAATCTAGCCG | SEQ ID NO: 108 |
| | CGATACGCTCGCAC*T*A | |
| CTL098_TOP_tag | /5Phos/A*C*CGCCGCTATACGCGCGATTAAGGTGTACGCTCGC | SEQ ID NO: 109 |
| | AGTCGCGGACTAAG*G*T | |
| CTL038_TOP_tag | /5Phos/T*A*CGCGCACTACTAACCGTCGAGTGCGTACGCTCGC | SEQ ID NO: 110 |
| | AGTACCGATCGCTA*G*T | |
| CTL139_TOP_tag | /5Phos/G*T*CGCGCAGTGTATAACAGCGCGTCGTTAGTGCGCG | SEQ ID NO: 111 |
| | AGAACGACGACTAG*G*T | |
| CTL010_TOP_tag | /5Phos/G*C*GTCGAATTGGTCGCGTAGTATGGTACCGCCGCTA | SEQ ID NO: 112 |
| | TACACCAATACGCC*G*C | |
| CTL034_TOP_tag | /5Phos/T*A*CGCGCACTACTTACGCGACTAGGTACCGATCGCT | SEQ ID NO: 113 |
| | AGTCGACGCGCTGT*T*A | |
| CTL117_TOP_tag | /5Phos/A*C*GCCGCTAACTATAGTTAGCGGCGTACCAATTCGA | SEQ ID NO: 114 |
| | CGCAACTAATCCGC*G*C | |
| CTL035_TOP_tag | /5Phos/C*G*CGGACTAAGGTTAGTTAGCGGCGTTACGCGCACT | SEQ ID NO: 115 |
| | ACTACCGATCCGCA*A*T | |
| CTL121_TOP_tag | /5Phos/A*C*GACGACTAGGTACCGCCGACTTATACGCCGCTAA | SEQ ID NO: 116 |
| | CTAATAGGTAGCGC*G*T | |
| CTL106_TOP_tag | /5Phos/C*G*GATCGACGGTTGCGCGAGTAGTGTAGTAGTACGC | SEQ ID NO: 117 |
| | GGTTACACTGCGCG*A*C | |
| CTL059_TOP_tag | /5Phos/A*T*TGCGGATCGGTACCGCCGACTTATACCGATCCGC | SEQ ID NO: 118 |
| | AATTCGCTCGATTG*G*T | |
| CTL157_TOP_tag | /5Phos/A*C*TGCGAGCGTACACTGCGAGCGTACACCTTAATCG | SEQ ID NO: 119 |
| | CGCACCGCTCGTTA*C*C | |
| CTL015_TOP_tag | /5Phos/A*C*TACTGTCGCGATCGTCGCACTAGTTACGCTCGCA | SEQ ID NO: 120 |
| | CTAATTGCGGATCG*G*T | |
| CTL110_TOP_tag | /5Phos/G*G*TAACGAGCGGTTCTCGCGCACTAATTAGTGCGCG | SEQ ID NO: 121 |
| | AGAACCATACTACG*C*G | |
| CTL123_TOP_tag | /5Phos/A*C*TACTCGCGCTAGCGCGATTAAGGTACCTTAATCG | SEQ ID NO: 122 |
| | CGCAACTACTCGCC*G*A | |
| CTL014_TOP_tag | /5Phos/T*A*CGCGCACTACTCTTGTACGGCGGTACCAATTCGA | SEQ ID NO: 123 |
| | CGCAACCGTCGAGT*G*C | |
| CTL131_TOP_tag | /5Phos/A*A*CCGTCGATCCGATTGCGGATCGGTACCTTAATCG | SEQ ID NO: 124 |
| | CGCACTAGTGCGAC*G*A | |
| CTL062_TOP_tag | /5Phos/A*G*TAGTGCGCGTATACACTGCGCGACACACTACTCG | SEQ ID NO: 125 |
| | CGCACCTTAATCCG*C*G | |
| CTL044_TOP_tag | /5Phos/A*C*GCCGTACCATACGCGGTAATAGGTAGTAGTGCGC | SEQ ID NO: 126 |
| | GTATTCGGCGCTAG*G*T | |
| CTL043_TOP_tag | /5Phos/T*A*GCGCGTCAAGAACCTAGCGTTGCGATAAGTCGGC | SEQ ID NO: 127 |
| | GGTAGTAGTACGCG*G*T | |
| CTL118_TOP_tag | /5Phos/C*G*CATTAGTCGGTAATCTAGCCGCGAACCATAACCG | SEQ ID NO: 128 |
| | CGCACCGATCGCTA*G*T | |
| CTL128_TOP_tag | /5Phos/T*A*TGGTACGGCGTGCGGCGTATTGGTACGCCGCTAA | SEQ ID NO: 129 |
| | CTAATAAGTCGGCG*G*T | |
| CTL067_TOP_tag | /5Phos/G*C*GCGGTTATGGTGCGGCGTATTGGTACGAGCGGTA | SEQ ID NO: 130 |
| | GTCAACCGCTCGTC*C*G | |
| CTL020_TOP_tag | /5Phos/C*G*ACTATGTGCGCAACTACTCGCCGAACCATAACCG | SEQ ID NO: 131 |
| | CGCTATGCGCTCGA*C*T | |
| CTL006_TOP_tag | /5Phos/T*A*GTTAGCGGCGTACCGCTCGTTACCACCTTAATCG | SEQ ID NO: 132 |
| | CGCACCATACTACG*C*G | |
| CTL017_TOP_tag | /5Phos/C*G*CATTAGTCGGTAGTAGTGCGCGTAAACCGCTCGT | SEQ ID NO: 133 |
| | CCGTTAGTGCGCGA*G*A | |
| CTL057_TOP_tag | /5Phos/T*A*GCGCGAGTAGTACCGACTAATGCGTCTCGCGCAC | SEQ ID NO: 134 |
| | TAAGACTACCGCTC*G*T | |
| CTL078_TOP_tag | /5Phos/T*A*CGCTCGCACTATCGCTCGATTGGTACCGCCGCTA | SEQ ID NO: 135 |
| | TACACCATAACCGC*G*C | |
| CTL031_TOP_tag | /5Phos/A*C*CAATCGAGCGAAGTCGAGCGCATAACGCGCTACC | SEQ ID NO: 136 |
| | TATACGCCGCTAAC*T*A | |
| CTL136_TOP_tag | /5Phos/A*C*CTTAATCCGCGACTGCGAGCGTACACCGACTAAT | SEQ ID NO: 137 |
| | GCGACTACTGTCGC*G*A | |
| CTL165_TOP_tag | /5Phos/A*G*TAGTGCGCGTATCGCTCGATTGGTTCTTGACGCG | SEQ ID NO: 138 |
| | CTAGTATAGCGGCG*G*T | |
| CTL039_TOP_tag | /5Phos/T*C*GTCGCACTAGTCGGTACGGTCGGTGCGCACATAG | SEQ ID NO: 139 |
| | TCGTATGGTACGGC*G*T | |
| CTL036_TOP_tag | /5Phos/C*G*CGGATTAAGGTAGTCGAGCGCATAACCGCGTACT | SEQ ID NO: 140 |
| | ACTACGACGACTAG*G*T | |
| CTL048_TOP_tag | /5Phos/C*G*ACTATGTGCGCTACGCTCGCACTAACACTACTCG | SEQ ID NO: 141 |
| | CGCACCTAGCGCCG*A*A | |
| CTL053_TOP_tag | /5Phos/A*C*CGCCGACTTATTCTCGCGCACTAATCGTCGCACT | SEQ ID NO: 142 |
| | AGTAACCGTCGATC*C*G | |
| CTL072_TOP_tag | /5Phos/A*C*CTAGCGTTGCGACCGACTAATGCGGGTAACGAGC | SEQ ID NO: 143 |
| | GGTTATGGTACGGC*G*T | |
| CTL096_TOP_tag | /5Phos/C*G*CGCTACTAGGTCGCGGTAATAGGTACCTAGCGTT | SEQ ID NO: 144 |
| | GCGACCTAGTCGCG*T*A | |
| CTL150_TOP_tag | /5Phos/C*G*TTCGGCTAGGTACTACTCGCGCTACGCATTAGTC | SEQ ID NO: 145 |
| | GGTTCGCGACAGTA*G*T | |
| CTL084_TOP_tag | /5Phos/C*G*GACGAGCGGTTCGCGGTAATAGGTACGACGACTA | SEQ ID NO: 146 |
| | GGTTAGTTAGCGGC*G*T | |
| CTL142_TOP_tag | /5Phos/T*A*CGCTCGCACTAATTGCGGATCGGTACCGACTAAT | SEQ ID NO: 147 |
| | GCGACCGCGTACTA*C*T | |
| CTL102_TOP_tag | /5Phos/A*C*CGACCGTACCGTATGGTACGGCGTTCTTGACGCG | SEQ ID NO: 148 |
| | CTAACCTAGCGCCG*A*A | |
| CTL154_TOP_tag | /5Phos/G*C*GCGGATTAGTTAACCGTCGAGTGCACACTACTCG | SEQ ID NO: 149 |
| | CGCACTGCGAGCGT*A*C | |
| CTL112_TOP_tag | /5Phos/A*C*CTTAATCCGCGACCGACTAATGCGTACGCGCACT | SEQ ID NO: 150 |
| | ACTATAAGTCGGCG*G*T | |
| CTL145_TOP_tag | /5Phos/A*C*CTTAATCCGCGGCGCGGTTATGGTACCGACTAAT | SEQ ID NO: 151 |
| | GCGAACCGCTCGTC*C*G | |
| CTL060_TOP_tag | /5Phos/A*C*TGCGAGCGTACCTTGTACGGCGGTACCTAGTAGC | SEQ ID NO: 152 |
| | GCGATAAGTCGGCG*G*T | |
| CTL016_TOP_tag | /5Phos/T*T*CGGCGCTAGGTACCTTAGTCCGCGTTCGGCGCTA | SEQ ID NO: 153 |
| | GGTACCTAGCGTTG*C*G | |
| CTL159_TOP_tag | /5Phos/A*C*CTAGTCGCGTACTTGTACGGCGGTACCTAGCCGA | SEQ ID NO: 154 |
| | ACGAACCGTCGAGT*G*C | |
| CTL056_TOP_tag | /5Phos/A*C*CATAACCGCGCTACACTGCGCGACACCAATACGC | SEQ ID NO: 155 |
| | CGCTATGGTACGGC*G*T | |
| CTL162_TOP_tag | /5Phos/A*C*ACTACTCGCGCTACGCGACTAGGTAATGTCGAAC | SEQ ID NO: 156 |
| | CGCACGCCGCTAAC*T*A | |
| CTL018_TOP_tag | /5Phos/A*C*CGACTAATGCGTAACAGCGCGTCGTTAGTGCGCG | SEQ ID NO: 157 |
| | AGAACCTTAATCGC*G*C | |
| CTL115_TOP_tag | /5Phos/A*C*GCCGTACCATAACCGACTAATGCGATAAGTCGGC | SEQ ID NO: 158 |
| | GGTACCAATACGCC*G*C | |
| CTL033_TOP_tag | /5Phos/G*T*ACGCTCGCAGTCGCGGTAATAGGTTCGGCGAGTA | SEQ ID NO: 159 |
| | GTTACCATAACCGC*G*C | |
| CTL047_TOP_tag | /5Phos/C*G*GACGAGCGGTTGCGCGGTTATGGTACTAGTGCGA | SEQ ID NO: 160 |
| | CGAGCGCACATAGT*C*G | |
| CTL108_TOP_tag | /5Phos/A*C*TACTCGCGCTAGCGCGATTAAGGTACGCCGCTAA | SEQ ID NO: 161 |
| | CTATCGCGGCTAGA*T*T | |
| CTL041_TOP_tag | /5Phos/A*C*CAATTCGACGCAACTAATCCGCGCACCAATTCGA | SEQ ID NO: 162 |
| | CGCAGTAGTGCGCG*T*A | |
| CTL061_TOP_tag | /5Phos/A*C*CGCCGCTATACACCTAGCGCCGAAGTACGCTCGC | SEQ ID NO: 163 |
| | AGTGTATAGCGGCG*G*T | |
| CTL166_TOP_tag | /5Phos/A*C*ACTACTCGCGCCGGACGAGCGGTTACCAATACGC | SEQ ID NO: 164 |
| | CGCTAGCGCGAGTA*G*T | |
| CTL012_TOP_tag | /5Phos/T*C*GTCGCACTAGTACCTTAATCCGCGCGCAACGCTA | SEQ ID NO: 165 |
| | GGTACACTACTCGC*G*C | |
| CTL052_TOP_tag | /5Phos/C*G*CGCTACTAGGTACCGACTAATGCGCGCAACGCTA | SEQ ID NO: 166 |
| | GGTAATGTCGAACC*G*C | |
| CTL153_TOP_tag | /5Phos/A*C*GAGCGGTAGTCACTACTGTCGCGACGCAACGCTA | SEQ ID NO: 167 |
| | GGTTACACTGCGCG*A*C | |
| CTL094_TOP_tag | /5Phos/A*C*CTAGTCGCGTACGCGTAGTATGGTACCGATCGCT | SEQ ID NO: 168 |
| | AGTGGTAACGAGCG*G*T | |
| CTL095_TOP_tag | /5Phos/G*C*GGTTCGACATTACCGACTAATGCGTATGCGCTCG | SEQ ID NO: 169 |
| | ACTACCTAGCGTTG*C*G | |
| CTL105_TOP_tag | /5Phos/A*C*TGCGAGCGTACTCTCGCGCACTAAACGCCGCTAA | SEQ ID NO: 170 |
| | CTACGCGCTACTAG*G*T | |
| CTL109_TOP_tag | /5Phos/C*G*GTACGGTCGGTAATCTAGCCGCGAACCTTAGTCC | SEQ ID NO: 171 |
| | GCGACCGCCGTACA*A*G | |
| CTL032_TOP_tag | /5Phos/T*C*GGCGAGTAGTTACGCGCTACCTATTCGCGGCTAG | SEQ ID NO: 172 |
| | ATTACGCCGCTAAC*T*A | |
| CTL161_BOT_tag | /5Phos/A*C*GCCGCTAACTAGCGCGATTAAGGTGTACGCTCGC | SEQ ID NO: 173 |
| | AGTGTCGCGCAGTG*T*A | |
| CTL164_BOT_tag | /5Phos/A*G*TAGTGCGCGTAGCGGTTCGACATTAGTAGTACGC | SEQ ID NO: 174 |
| | GGTGCACTCGACGG*T*T | |
| CTL030_BOT_tag | /5Phos/T*C*GCGGCTAGATTAGTAGTGCGCGTAACACTACTCG | SEQ ID NO: 175 |
| | CGCACCTTAGTCCG*C*G | |
| CTL088_BOT_tag | /5Phos/A*C*TAGCGATCGGTGCGTCGAATTGGTTAGCGCGAGT | SEQ ID NO: 176 |
| | AGTTCGTCGCACTA*G*T | |
| CTL148_BOT_tag | /5Phos/C*G*CGGACTAAGGTGCGCGGTTATGGTACACTACTCG | SEQ ID NO: 177 |
| | CGCGCGGTTCGACA*T*T | |
| CTL152_BOT_tag | /5Phos/A*C*GCGCTACCTATGCGGCGTATTGGTATAAGTCGGC | SEQ ID NO: 178 |
| | GGTACCAATTCGAC*G*C | |
| CTL007_BOT_tag | /5Phos/A*C*CATACTACGCGTTGTCGCGCTAGTACCAATTCGA | SEQ ID NO: 179 |
| | CGCCGCGCTACTAG*G*T | |
| CTL141_BOT_tag | /5Phos/A*C*CGACCGTACCGTAGTTAGCGGCGTACCTTAATCG | SEQ ID NO: 180 |
| | CGCGGTAACGAGCG*G*T | |
| CTL064_BOT_tag | /5Phos/G*T*ACGCTCGCAGTGCGTCGAATTGGTACCTAGCCGA | SEQ ID NO: 181 |
| | ACGATAAGTCGGCG*G*T | |
| CTL158_BOT_tag | /5Phos/T*A*ACAGCGCGTCGCGCGGTAATAGGTGTACGCTCGC | SEQ ID NO: 182 |
| | AGTCGCGGATTAAG*G*T | |
| CTL066_BOT_tag | /5Phos/G*C*GTCGAATTGGTTAGCGCGTCAAGAGGTAACGAGC | SEQ ID NO: 183 |
| | GGTACCTAGTCGTC*G*T | |
| CTL144_BOT_tag | /5Phos/T*A*CGCTCGCACTAGCGCGGTTATGGTAATGTCGAAC | SEQ ID NO: 184 |
| | CGCCGCGTAGTATG*G*T | |
| CTL107_BOT_tag | /5Phos/A*C*TAGTGCGACGAGCGGCGTATTGGTACCAATACGC | SEQ ID NO: 185 |
| | CGCACCGCCGTACA*A*G | |
| CTL149_BOT_tag | /5Phos/T*T*GTCGCGCTAGTGCGCGATTAAGGTATAAGTCGGC | SEQ ID NO: 186 |
| | GGTACTGCGAGCGT*A*C | |
| CTL008_BOT_tag | /5Phos/C*G*CGGACTAAGGTACTACTCGCGCTAACGCCGTACC | SEQ ID NO: 187 |
| | ATAACCTAGTCGTC*G*T | |
| CTL099_BOT_tag | /5Phos/A*C*TAGCGATCGGTTAGCGCGTCAAGAACGCGCTACC | SEQ ID NO: 188 |
| | TATGACTACCGCTC*G*T | |
| CTL089_BOT_tag | /5Phos/C*T*TGTACGGCGGTGCGCGGTTATGGTACCAATTCGA | SEQ ID NO: 189 |
| | CGCATTGCGGATCG*G*T | |
| CTL081_BOT_tag | /5Phos/T*C*GCTCGATTGGTCGCGGTAATAGGTTCGCGACAGT | SEQ ID NO: 190 |
| | AGTTCGTCGCACTA*G*T | |
| CTL075_BOT_tag | /5Phos/A*C*CTAGCGCCGAACGGACGAGCGGTTACTACTGTCG | SEQ ID NO: 191 |
| | CGACTTGTACGGCG*G*T | |
| CTL160_BOT_tag | /5Phos/A*C*GCGCTACCTATACCGCGTACTACTACCGACTAAT | SEQ ID NO: 192 |
| | GCGACTAGTGCGAC*G*A | |
| CTL133_BOT_tag | /5Phos/A*A*CCGTCGAGTGCGCGCGAGTAGTGTACGCCGCTAA | SEQ ID NO: 193 |
| | CTAGCGTCGAATTG*G*T | |
| CTL076_BOT_tag | /5Phos/G*C*GCGAGTAGTGTGACTACCGCTCGTACCTATTACC | SEQ ID NO: 194 |
| | GCGACCTATTACCG*C*G | |
| CTL024_BOT_tag | /5Phos/T*C*GCTCGATTGGTTACGCGCACTACTTACGCTCGCA | SEQ ID NO: 195 |
| | CTAAACTACTCGCC*G*A | |
| CTL045_BOT_tag | /5Phos/T*C*GTCGCACTAGTGCGCGGTTATGGTACCATAACCG | SEQ ID NO: 196 |
| | CGCTACACTGCGCG*A*C | |
| CTL009_BOT_tag | /5Phos/A*C*CGCGTACTACTGCGGTTCGACATTACCTTAATCG | SEQ ID NO: 197 |
| | CGCAGTCGAGCGCA*T*A | |
| CTL055_BOT_tag | /5Phos/A*G*TAGTGCGCGTAGCGTCGAATTGGTGCGCACATAG | SEQ ID NO: 198 |
| | TCGTTGTCGCGCTA*G*T | |
| CTL101_BOT_tag | /5Phos/G*C*GCGGATTAGTTGCGTCGAATTGGTACCGCCGTAC | SEQ ID NO: 199 |
| | AAGTCGGCGAGTAG*T*T | |
| CTL135_BOT_tag | /5Phos/A*G*TAGTGCGCGTACGTTCGGCTAGGTACCGCCGTAC | SEQ ID NO: 200 |
| | AAGACCTTAATCCG*C*G | |
| CTL155_BOT_tag | /5Phos/A*A*CCGTCGAGTGCATTGCGGATCGGTACCGCCGTAC | SEQ ID NO: 201 |
| | AAGTCTTGACGCGC*T*A | |
| CTL122_BOT_tag | /5Phos/G*C*GGCGTATTGGTACCTAGTCGTCGTACCAATACGC | SEQ ID NO: 202 |
| | CGCACCGACTAATG*C*G | |
| CTL080_BOT_tag | /5Phos/A*C*CGATCGCTAGTCGCATTAGTCGGTACCATAACCG | SEQ ID NO: 203 |
| | CGCCGCGCTACTAG*G*T | |
| CTL126_BOT_tag | /5Phos/T*A*GTGCGAGCGTATCGCGGCTAGATTACGACGACTA | SEQ ID NO: 204 |
| | GGTTAGCGCGAGTA*G*T | |
| CTL098_BOT_tag | /5Phos/A*C*CTTAGTCCGCGACTGCGAGCGTACACCTTAATCG | SEQ ID NO: 205 |
| | CGCGTATAGCGGCG*G*T | |
| CTL038_BOT_tag | /5Phos/A*C*TAGCGATCGGTACTGCGAGCGTACGCACTCGACG | SEQ ID NO: 206 |
| | GTTAGTAGTGCGCG*T*A | |
| CTL139_BOT_tag | /5Phos/A*C*CTAGTCGTCGTTCTCGCGCACTAACGACGCGCTG | SEQ ID NO: 207 |
| | TTATACACTGCGCG*A*C | |
| CTL010_BOT_tag | /5Phos/G*C*GGCGTATTGGTGTATAGCGGCGGTACCATACTAC | SEQ ID NO: 208 |
| | GCGACCAATTCGAC*G*C | |
| CTL034_BOT_tag | /5Phos/T*A*ACAGCGCGTCGACTAGCGATCGGTACCTAGTCGC | SEQ ID NO: 209 |
| | GTAAGTAGTGCGCG*T*A | |
| CTL117_BOT_tag | /5Phos/G*C*GCGGATTAGTTGCGTCGAATTGGTACGCCGCTAA | SEQ ID NO: 210 |
| | CTATAGTTAGCGGC*G*T | |
| CTL035_BOT_tag | /5Phos/A*T*TGCGGATCGGTAGTAGTGCGCGTAACGCCGCTAA | SEQ ID NO: 211 |
| | CTAACCTTAGTCCG*C*G | |
| CTL121_BOT_tag | /5Phos/A*C*GCGCTACCTATTAGTTAGCGGCGTATAAGTCGGC | SEQ ID NO: 212 |
| | GGTACCTAGTCGTC*G*T | |
| CTL106_BOT_tag | /5Phos/G*T*CGCGCAGTGTAACCGCGTACTACTACACTACTCG | SEQ ID NO: 213 |
| | CGCAACCGTCGATC*C*G | |
| CTL059_BOT_tag | /5Phos/A*C*CAATCGAGCGAATTGCGGATCGGTATAAGTCGGC | SEQ ID NO: 214 |
| | GGTACCGATCCGCA*A*T | |
| CTL157_BOT_tag | /5Phos/G*G*TAACGAGCGGTGCGCGATTAAGGTGTACGCTCGC | SEQ ID NO: 215 |
| | AGTGTACGCTCGCA*G*T | |
| CTL015_BOT_tag | /5Phos/A*C*CGATCCGCAATTAGTGCGAGCGTAACTAGTGCGA | SEQ ID NO: 216 |
| | CGATCGCGACAGTA*G*T | |
| CTL110_BOT_tag | /5Phos/C*G*CGTAGTATGGTTCTCGCGCACTAATTAGTGCGCG | SEQ ID NO: 217 |
| | AGAACCGCTCGTTA*C*C | |
| CTL123_BOT_tag | /5Phos/T*C*GGCGAGTAGTTGCGCGATTAAGGTACCTTAATCG | SEQ ID NO: 218 |
| | CGCTAGCGCGAGTA*G*T | |
| CTL014_BOT_tag | /5Phos/G*C*ACTCGACGGTTGCGTCGAATTGGTACCGCCGTAC | SEQ ID NO: 219 |
| | AAGAGTAGTGCGCG*T*A | |
| CTL131_BOT_tag | /5Phos/T*C*GTCGCACTAGTGCGCGATTAAGGTACCGATCCGC | SEQ ID NO: 220 |
| | AATCGGATCGACGG*T*T | |
| CTL062_BOT_tag | /5Phos/C*G*CGGATTAAGGTGCGCGAGTAGTGTGTCGCGCAGT | SEQ ID NO: 221 |
| | GTATACGCGCACTA*C*T | |
| CTL044_BOT_tag | /5Phos/A*C*CTAGCGCCGAATACGCGCACTACTACCTATTACC | SEQ ID NO: 222 |
| | GCGTATGGTACGGC*G*T | |
| CTL043_BOT_tag | /5Phos/A*C*CGCGTACTACTACCGCCGACTTATCGCAACGCTA | SEQ ID NO: 223 |
| | GGTTCTTGACGCGC*T*A | |
| CTL118_BOT_tag | /5Phos/A*C*TAGCGATCGGTGCGCGGTTATGGTTCGCGGCTAG | SEQ ID NO: 224 |
| | ATTACCGACTAATG*C*G | |
| CTL128_BOT_tag | /5Phos/A*C*CGCCGACTTATTAGTTAGCGGCGTACCAATACGC | SEQ ID NO: 225 |
| | CGCACGCCGTACCA*T*A | |
| CTL067_BOT_tag | /5Phos/C*G*GACGAGCGGTTGACTACCGCTCGTACCAATACGC | SEQ ID NO: 226 |
| | CGCACCATAACCGC*G*C | |
| CTL020_BOT_tag | /5Phos/A*G*TCGAGCGCATAGCGCGGTTATGGTTCGGCGAGTA | SEQ ID NO: 227 |
| | GTTGCGCACATAGT*C*G | |
| CTL006_BOT_tag | /5Phos/C*G*CGTAGTATGGTGCGCGATTAAGGTGGTAACGAGC | SEQ ID NO: 228 |
| | GGTACGCCGCTAAC*T*A | |
| CTL017_BOT_tag | /5Phos/T*C*TCGCGCACTAACGGACGAGCGGTTTACGCGCACT | SEQ ID NO: 229 |
| | ACTACCGACTAATG*C*G | |
| CTL057_BOT_tag | /5Phos/A*C*GAGCGGTAGTCTTAGTGCGCGAGACGCATTAGTC | SEQ ID NO: 230 |
| | GGTACTACTCGCGC*T*A | |
| CTL078_BOT_tag | /5Phos/G*C*GCGGTTATGGTGTATAGCGGCGGTACCAATCGAG | SEQ ID NO: 231 |
| | CGATAGTGCGAGCG*T*A | |
| CTL031_BOT_tag | /5Phos/T*A*GTTAGCGGCGTATAGGTAGCGCGTTATGCGCTCG | SEQ ID NO: 232 |
| | ACTTCGCTCGATTG*G*T | |
| CTL136_BOT_tag | /5Phos/T*C*GCGACAGTAGTCGCATTAGTCGGTGTACGCTCGC | SEQ ID NO: 233 |
| | AGTCGCGGATTAAG*G*T | |
| CTL165_BOT_tag | /5Phos/A*C*CGCCGCTATACTAGCGCGTCAAGAACCAATCGAG | SEQ ID NO: 234 |
| | CGATACGCGCACTA*C*T | |
| CTL039_BOT_tag | /5Phos/A*C*GCCGTACCATACGACTATGTGCGCACCGACCGTA | SEQ ID NO: 235 |
| | CCGACTAGTGCGAC*G*A | |
| CTL036_BOT_tag | /5Phos/A*C*CTAGTCGTCGTAGTAGTACGCGGTTATGCGCTCG | SEQ ID NO: 236 |
| | ACTACCTTAATCCG*C*G | |
| CTL048_BOT_tag | /5Phos/T*T*CGGCGCTAGGTGCGCGAGTAGTGTTAGTGCGAGC | SEQ ID NO: 237 |
| | GTAGCGCACATAGT*C*G | |
| CTL053_BOT_tag | /5Phos/C*G*GATCGACGGTTACTAGTGCGACGATTAGTGCGCG | SEQ ID NO: 238 |
| | AGAATAAGTCGGCG*G*T | |
| CTL072_BOT_tag | /5Phos/A*C*GCCGTACCATAACCGCTCGTTACCCGCATTAGTC | SEQ ID NO: 239 |
| | GGTCGCAACGCTAG*G*T | |
| CTL096_BOT_tag | /5Phos/T*A*CGCGACTAGGTCGCAACGCTAGGTACCTATTACC | SEQ ID NO: 240 |
| | GCGACCTAGTAGCG*C*G | |
| CTL150_BOT_tag | /5Phos/A*C*TACTGTCGCGAACCGACTAATGCGTAGCGCGAGT | SEQ ID NO: 241 |
| | AGTACCTAGCCGAA*C*G | |
| CTL084_BOT_tag | /5Phos/A*C*GCCGCTAACTAACCTAGTCGTCGTACCTATTACC | SEQ ID NO: 242 |
| | GCGAACCGCTCGTC*C*G | |
| CTL142_BOT_tag | /5Phos/A*G*TAGTACGCGGTCGCATTAGTCGGTACCGATCCGC | SEQ ID NO: 243 |
| | AATTAGTGCGAGCG*T*A | |
| CTL102_BOT_tag | /5Phos/T*T*CGGCGCTAGGTTAGCGCGTCAAGAACGCCGTACC | SEQ ID NO: 244 |
| | ATACGGTACGGTCG*G*T | |
| CTL154_BOT_tag | /5Phos/G*T*ACGCTCGCAGTGCGCGAGTAGTGTGCACTCGACG | SEQ ID NO: 245 |
| | GTTAACTAATCCGC*G*C | |
| CTL112_BOT_tag | /5Phos/A*C*CGCCGACTTATAGTAGTGCGCGTACGCATTAGTC | SEQ ID NO: 246 |
| | GGTCGCGGATTAAG*G*T | |
| CTL145_BOT_tag | /5Phos/C*G*GACGAGCGGTTCGCATTAGTCGGTACCATAACCG | SEQ ID NO: 247 |
| | CGCCGCGGATTAAG*G*T | |
| CTL060_BOT_tag | /5Phos/A*C*CGCCGACTTATCGCGCTACTAGGTACCGCCGTAC | SEQ ID NO: 248 |
| | AAGGTACGCTCGCA*G*T | |
| CTL016_BOT_tag | /5Phos/C*G*CAACGCTAGGTACCTAGCGCCGAACGCGGACTAA | SEQ ID NO: 249 |
| | GGTACCTAGCGCCG*A*A | |
| CTL159_BOT_tag | /5Phos/G*C*ACTCGACGGTTCGTTCGGCTAGGTACCGCCGTAC | SEQ ID NO: 250 |
| | AAGTACGCGACTAG*G*T | |
| CTL056_BOT_tag | /5Phos/A*C*GCCGTACCATAGCGGCGTATTGGTGTCGCGCAGT | SEQ ID NO: 251 |
| | GTAGCGCGGTTATG*G*T | |
| CTL162_BOT_tag | /5Phos/T*A*GTTAGCGGCGTGCGGTTCGACATTACCTAGTCGC | SEQ ID NO: 252 |
| | GTAGCGCGAGTAGT*G*T | |
| CTL018_BOT_tag | /5Phos/G*C*GCGATTAAGGTTCTCGCGCACTAACGACGCGCTG | SEQ ID NO: 253 |
| | TTACGCATTAGTCG*G*T | |
| CTL115_BOT_tag | /5Phos/G*C*GGCGTATTGGTACCGCCGACTTATCGCATTAGTC | SEQ ID NO: 254 |
| | GGTTATGGTACGGC*G*T | |
| CTL033_BOT_tag | /5Phos/G*C*GCGGTTATGGTAACTACTCGCCGAACCTATTACC | SEQ ID NO: 255 |
| | GCGACTGCGAGCGT*A*C | |
| CTL047_BOT_tag | /5Phos/C*G*ACTATGTGCGCTCGTCGCACTAGTACCATAACCG | SEQ ID NO: 256 |
| | CGCAACCGCTCGTC*C*G | |
| CTL108_BOT_tag | /5Phos/A*A*TCTAGCCGCGATAGTTAGCGGCGTACCTTAATCG | SEQ ID NO: 257 |
| | CGCTAGCGCGAGTA*G*T | |
| CTL041_BOT_tag | /5Phos/T*A*CGCGCACTACTGCGTCGAATTGGTGCGCGGATTA | SEQ ID NO: 258 |
| | GTTGCGTCGAATTG*G*T | |
| CTL061_BOT_tag | /5Phos/A*C*CGCCGCTATACACTGCGAGCGTACTTCGGCGCTA | SEQ ID NO: 259 |
| | GGTGTATAGCGGCG*G*T | |
| CTL166_BOT_tag | /5Phos/A*C*TACTCGCGCTAGCGGCGTATTGGTAACCGCTCGT | SEQ ID NO: 260 |
| | CCGGCGCGAGTAGT*G*T | |
| CTL012_BOT_tag | /5Phos/G*C*GCGAGTAGTGTACCTAGCGTTGCGCGCGGATTAA | SEQ ID NO: 261 |
| | GGTACTAGTGCGAC*G*A | |
| CTL052_BOT_tag | /5Phos/G*C*GGTTCGACATTACCTAGCGTTGCGCGCATTAGTC | SEQ ID NO: 262 |
| | GGTACCTAGTAGCG*C*G | |
| CTL153_BOT_tag | /5Phos/G*T*CGCGCAGTGTAACCTAGCGTTGCGTCGCGACAGT | SEQ ID NO: 263 |
| | AGTGACTACCGCTC*G*T | |
| CTL094_BOT_tag | /5Phos/A*C*CGCTCGTTACCACTAGCGATCGGTACCATACTAC | SEQ ID NO: 264 |
| | GCGTACGCGACTAG*G*T | |
| CTL095_BOT_tag | /5Phos/C*G*CAACGCTAGGTAGTCGAGCGCATACGCATTAGTC | SEQ ID NO: 265 |
| | GGTAATGTCGAACC*G*C | |
| CTL105_BOT_tag | /5Phos/A*C*CTAGTAGCGCGTAGTTAGCGGCGTTTAGTGCGCG | SEQ ID NO: 266 |
| | AGAGTACGCT CGCA*G*T | |
| CTL109_BOT_tag | /5Phos/C*T*TGTACGGCGGTCGCGGACTAAGGTTCGCGGCTAG | SEQ ID NO: 267 |
| | ATTACCGACCGTAC*C*G | |
| CTL032_BOT_tag | /5Phos/T*A*GTTAGCGGCGTAATCTAGCCGCGAATAGGTAGCG | SEQ ID NO: 268 |
| | CGTAACTACTCGCC*G*A | |

“/5Phos/” indicates a 5′-phosphate moiety; “*” indicates a phosphorothioate linkage.

TABLE 5
Pools of Tag Sequences
Pools

| Tags | Pool A1 | Pool B1 | Pool B2 | Pool B3 | Pool B4 | Pool B5 | Pool B6 | Pool C1 |
|---|---|---|---|---|---|---|---|---|
| Present in | CTL085 | CTL161 | CTL089 | CTL098 | CTL062 | CTL048 | CTL018 | Pool A1 |
| Pools | CTL169 | CTL164 | CTL081 | CTL038 | CTL044 | CTL053 | CTL115 | Pool B1 |
| | CTL137 | CTL030 | CTL075 | CTL139 | CTL043 | CTL072 | CTL033 | Pool B2 |
| | CTL042 | CTL088 | CTL160 | CTL010 | CTL118 | CTL096 | CTL047 | Pool B3 |
| | CTL051 | CTL148 | CTL133 | CTL034 | CTL128 | CTL150 | CTL108 | Pool B4 |
| | CTL167 | CTL152 | CTL076 | CTL117 | CTL067 | CTL084 | CTL041 | Pool B5 |
| | CTL026 | CTL007 | CTL024 | CTL035 | CTL020 | CTL142 | CTL061 | Pool B6 |
| | CTL068 | CTL141 | CTL045 | CTL121 | CTL006 | CTL102 | CTL166 | |
| | CTL138 | CTL064 | CTL009 | CTL106 | CTL017 | CTL154 | CTL012 | |
| | CTL079 | CTL158 | CTL055 | CTL059 | CTL057 | 0TL112 | CTL052 | |
| | CTL063 | CTL066 | CTL101 | CTL157 | CTL078 | 0TL145 | CTL153 | |
| | CTL168 | CTL144 | CTL135 | CTL015 | CTL031 | CTL060 | CTL094 | |
| | CTL021 | CTL107 | CTL155 | CTL110 | CTL136 | CTL016 | CTL095 | |
| | CTL151 | CTL149 | CTL122 | CTL123 | CTL165 | CTL159 | CTL105 | |
| | CTL002 | CTL008 | CTL080 | CTL014 | CTL039 | CTL056 | CTL109 | |
| | CTL134 | CTL099 | CTL126 | CTL131 | CTL036 | CTL162 | CTL032 | |

TABLE 6
Non-homologous tails

| Name | Sequence (5′→3′) | SEQ ID NO: |
|---|---|---|
| H1 | ACGCGACTATACGCGCAATATGGT | SEQ ID NO: 269 |
| H2 | CTAGCGATACTACGCGATACGAGAT | SEQ ID NO: 270 |
| H3 | CATAGCGGTATTACGCGAGATTACGA | SEQ ID NO: 271 |
| H4 | CGCGAGTACGTACGATTACCG | SEQ ID NO: 272 |
| H5 | ACGCGCGACTATACGCGCCTC | SEQ ID NO: 273 |

What is claimed:

1. A method for identifying and nominating on- and off-target CRISPR edited sites with improved accuracy and sensitivity, the process comprising the steps of:
(a) co-delivering a guide sequence RNA (sgRNA) or a two-part CRISPR RNA:trans-activating crRNA (crRNA:tracrRNA) duplex, one or more tag sequences, and an RNA-guided endonuclease to cells;
(b) incubating the cells for a period of time sufficient for double strand breaks to occur;
(c) isolating genomic DNA from the cells, fragmenting the genomic DNA, and ligating the fragmented genomic DNA to a unique molecular index containing a universal adapter sequence;
(d) amplifying the ligated DNA fragments using primers targeting the tag and universal adapter sequences to produce a first set of amplified sequences;
(e) amplifying the first set of amplified sequences using universal sequencing primers targeting the tails of Tag-pTOP or Tag-pBOT primers to produce a second set of amplified sequences;
(f) sequencing the pooled sequences and obtaining sequencing data; and
(g) identifying on-/off-target CRISPR editing loci.

2. The method of claim 1, wherein the universal sequencing primers target SP1 or SP2 sequence (SEQ ID NO: 7, 8) tails on the Tag-pTOP or Tag-pBOT primers to produce a second set of amplified sequences.

3. The method of claim 1, wherein the universal sequencing primers target predesigned non-homologous sequence (SEQ ID NO: 269-273) tails on the Tag-pTOP or Tag-pBot primers to produce a second set of amplified sequences.

4. The method of claim 1, wherein the universal sequencing primers target predesigned 13-mer tails on the Tag-pTOP or Tag-pBot primers to produce a second set of amplified sequences.

5. The method of claim 1, wherein step (g) comprises executing on a processor:
aligning the sequence data to a reference genome;
(ii) identifying on-/off-target CRISPR editing loci; and
(iii) outputting the alignment, analysis, and results data as custom-formatted files, tables or graphics.

6. The method of claim 1, further comprising a step following step (e) comprising:
(e1) normalizing the second set of amplified sequences to produce concentration normalized libraries, pooling the normalized libraries with other samples to produce pooled libraries; and continuing with steps (f)-(i).

7. The method of claim 1, wherein step (d) uses a supression PCR method.

8. The method of claim 1, wherein the RNA-guided endonuclease comprises an endogenously-expressed Cas enzyme, a Cas expression vector, a Cas protein, or a Cas RNP complex.

9. The method of claim 1, wherein the RNA-guided endonuclease comprises an endogenously-expressed Cas9 enzyme, a Cas9 expression vector, a Cas9 protein, or a Cas9 RNP complex.

10. The method of claim 1, wherein the cells comprise human or mouse cells.

11. The method of claim 1, wherein the period of time is about 24 hours to about 96 hours.

12. The method of claim 1, wherein multiple tag sequences are co-delivered.

13. The method of claim 1, wherein the tag sequences comprise double-stranded deoxyribooligonucleotides (dsDNA) comprising 52-base pairs.

14. The method of claim 1, wherein the tag sequences comprise a 5′-terminal phosphate, and phosphorothioate linkages between the 1^{st} and 2^{nd}, 2^{nd} and 3^{rd}, 50^{th} and 51^{st}, and 51^{st} and 52^{nd} nucleotides.

15. The method of claim 1, wherein the tag sequences comprise a double stranded DNA comprising the complementary top and bottom strand pairs of SEQ ID NO: 1-2 or 7-268.

16. On- and off-target CRISPR editing sites identified or nominated using the method of claim 1.

17. A method for designing 52-base pair tag sequences, the method comprising, executing on a processor:
(a) randomly generating 13-nucleotide sequences with 40-90% GC content, max homopolymer length A:2, C:3, G:2, T:2, weighted homopolymer rate <20, self-folding Tₘ<50° C., and self-dimer Tₘ<50° C.;
(b) removing sequences that perfectly align to a particular genome or that are homopolymers or GG or CC dinucleotide motifs and obtaining a set of 13-mers;
(c) selecting a subset of the 13-mer sequences that contain one or less CC or GG dinucleotide motifs;
(d) concatenating four of the of 13-mer subset sequences to form random 52-mer sequences;
(e) aligning the random 52-mer sequences to a genome;
(f) removing the random 52-mer sequences that have similarity to the genome to produce a subset of 52-mer sequences; and
(h) outputting the subset of 52-mer sequences and generating the complementary strands to produce double stranded 52-base pair tag sequences.

18. The method of claim 17, wherein the genome is human or mouse.

19. The method of claim 17, wherein the 52-base pair tag sequences are-non complementary to the genome.

20. The method of claim 17, further comprising designing primers for the 52-base pair tag sequences.

21. The method of claim 17, wherein the 52-base pair tag sequences comprise a 5′-terminal phosphate, and phosphorothioate linkages between the 1^{st} and 2^{nd}, 2^{nd} and 3^{rd}, 50^{th} and 51^{st}, and 51^{st} and 52^{nd} nucleotides of the 52-base pair tag sequences.

22. The method of claim 17, further comprising synthesizing oligonucleotides comprising the 52-base pair tag sequences, the complement of the 52-base pair tag sequences, or primers for the 52-base pair tag sequences.

23. One or more 52-base pair tag sequences designed using the methods of claim 17.

24. The 52-base pair tag sequences of claim 23, wherein the 52-base pair tag sequence comprises a double stranded DNA comprising the top and bottom strand pairs of SEQ ID NO: 1-2 or 7-268.

25. A method for designing primers partially complementary to the 52-base pair tag sequences of claim 23 and an adapter primer, the method comprising, executing on a processor:
(a) designing tag primers that are partially complementary to the top and bottom strands of tag sequences; and
(b) designing an adapter primer that is partially complementary to the top strand of the adapter sequence;
wherein:
the tag primers comprise a 5′-universal tail sequence; and
the adapter primer comprises a sequence complementary to the tails of Tag-pTOP or Tag-pBOT primers.

26. The method of claim 25, wherein the 5′-universal tail sequence is complementary to an SP1 or SP2 sequence (SEQ ID NO: 7, 8), a locus specific segment, a ribonucleotide (rN) 6-nucleotides from the 3′-end, a 3′-end mismatch, a 3′-end block (3′-C₃ spacer), a predesigned non-homologous sequence (SEQ ID NO: 269-273), or a predesigned 13-mer sequence.

27. The method of claim 25, wherein the primers partially complementary to top and bottom strands of the tag sequences comprise a tail sequence complementary to the SP1 sequence (SEQ ID NO: 7) and the adapter primer comprises a sequence complementary to the SP2 sequence (SEQ ID NO: 8) tail on the Tag-pTOP or Tag-pBOT primers; or the primers partially complementary to top and bottom strands of the tag sequences comprise a tail sequence complementary to the SP2 sequence (SEQ ID NO: 8) and the adapter primer comprises a sequence complementary to the SP1 sequence (SEQ ID NO: 7) tail on the Tag-pTOP or Tag-pBOT primers.

28. The method of claim 25, wherein the amplification of a nucleic acid molecule with the primers that are complementary to the top and bottom strands of tag sequences and primers that are complementary to the top strand of the adapter sequence produces a PCR product that comprises a portion of the tag sequence, a sgDNA sequence, and the adapter sequence.

29. The method of claim 25, further comprising synthesizing oligonucleotides comprising the sequences of the forward and reverse tag primers and the adapter primer.

30. The method of claim 25, wherein the 52-base pair tag sequences and primers partially complementary to the 52-base pair tag sequences are designed and selected using an algorithm predicting whether the primers are likely to be partially complementary and have a propensity to form primer-dimers.

31. One or more primers partially complementary to the 52-base pair tag sequences and one or more adapter primers designed using the method of claim 25.

32. The primers of claim 31, wherein the primers comprise the sequences of SEQ ID NO: 3, 4; and the adapter primer, wherein the adapter primer comprises the sequence of SEQ ID NO: 5.

33. A method for using of one or more double-stranded 52-base pair tag sequences to identify on- and off-target CRISPR editing sites.
